(19) United States
(12) Patent Application Publication
(10) Pub. No.: US 2019/0133124 A1
(43) Pub. Date: May 9, 2019

(54) BACILLUS AND LIPOCHITOOLIGOSACCHARIDE FOR IMPROVING PLANT GROWTH

(71) Applicant: NOVOZYMES BIOAG A/S, Bagsvaerd (DK)

(72) Inventors: Cassandra Marin, Roanoke, VA (US); Ahsan Habib, Roanoke, VA (US); Yaowei Kang, Chapel Hill, NC (US)

(73) Assignee: NOVOZYMES BIOAG A/S, Bagsvaerd (DK)

(21) Appl. No.: 16/304,339

(22) PCT Filed: May 22, 2017
(86) PCT No.: PCT/US2017/033778

§ 371 Date: Nov. 26, 2018

Related U.S. Application Data
(60) Provisional application No. 62/341,930, filed on May 26, 2016.

(51) Int. Cl. A01N 43/16; C12N 1/20

(52) U.S. Cl. CPC A01N 43/16; C12N 1/20

(57) ABSTRACT

Disclosed are compositions and methods for improving plant growth. In one example, the compositions contain Bacillus amyloliquefaciens bacteria and at least one lipochitooligosaccharide (LCO), and may be applied to plant or seed to improve growth and/or yield. In one example, the improvement in plant growth is more than additive as compared to application of either the bacteria or LCO alone.

### REFERENCE TO A DEPOSIT OF BIOLOGICAL MATERIAL

This application contains a reference to a deposit of biological material, which deposit is incorporated herein by reference. For complete information see the last paragraph of the description.

### BACKGROUND

Plant growth may be facilitated in a variety of ways. In one example, certain microbes can improve plant growth. Some “plant growth-promoting bacteria” (PGPB) may kill or inhibit plant pathogens (e.g., biocontrol activity). Some PGPB may improve plant growth through biocontrol-independent activities (e.g., by increasing nutrient availability to plants).

In addition to microbes, certain molecules produced by microbes may improve plant growth. In one example, lipochitooligosaccharides (LCDs) are microbe-produced compounds that are generally thought of as signaling molecules that facilitate a symbiotic relationship between certain microbes and plants. Nod factors are LCOs generally produced by bacteria known as Rhizobia. Myc factors are LCOs produced by Mycorrhizal fungi.

Although there are a variety of ways to improve plant growth using microbes and related molecules, new ways of doing this are needed.

### SUMMARY

We found that a combination treatment of plants with both a pure culture of Bacillus amyloliquefaciens and a lipochitooligosaccharide (LCO) promotes a more than additive improvement in plant growth which is greater than the additive effect of either Bacillus amyloliquefaciens or LCO treatment of the plant alone. That improvement in plant growth by treating plants/seeds includes, but is not limited to: an increase in root and/or shoot length; increases in dry weight of the roots and/or shoots, and/or total plant biomass; an increase in number of roots and/or shoots; an increase in germination frequency; and a decrease in the time taken for germination. In one example, the Bacillus amyloliquefaciens is strain SB 3281 (deposited as PTA-7542). In one example, the LCO is a Nod factor or Myc factor. In one example, the plant is corn. In one example, seeds of the plant are treated with the Bacillus amyloliquefaciens/LCO combination.

The more than additive effect of the combination of Bacillus amyloliquefaciens and an LCO on plant growth is unexpected. LCOs are oligosaccharides and can be utilized as carbon sources by microbes like bacteria and fungi. It was believed that an organism like Bacillus amyloliquefaciens might use LCO as a carbon source. It was thought likely that, in seed treatments combining both Bacillus amyloliquefaciens and LCOs, that an effect on seed germination and subsequent seedling growth facilitated by the LCOs could be reduced or lost due to metabolism of the LCO in presence of the bacteria.

Also, although some Bacillus amyloliquefaciens strains do have one or both of biocontrol activities and biocontrol-independent plant growth-facilitating activities, those activities are thought to be independent of LCOs. LCOs are also thought to be independent of Bacillus amyloliquefaciens—LCOs generally initiate and improve nodulation of nitrogen fixing bacteria on plant roots. Since the plant growth-facilitating activities attributed to either Bacillus amyloliquefaciens or LCOs are not thought to be mechanistically linked (i.e., they work independently of each other), it was not expected that a combined seed treatment with both Bacillus amyloliquefaciens and an LCO would improve plant growth in a more than additive fashion, or possibly even additively, as compared to their effects alone.

In a first aspect of the invention, compositions of an isolated and biologically pure culture of Bacillus amyloliquefaciens and at least one LCO are disclosed. In a second aspect, the compositions are used for improving plant growth, as compared to growth of plants on which the compositions have not been used.

In a third aspect, methods of improving plant growth by applying to a seed, supplying to a seed, contacting a seed with, or exposing a seed to, a composition of Bacillus amyloliquefaciens and at least one LCO are disclosed. The composition may also be applied to a furrow in which seeds are planted. The methods of improving plant growth also include applying to a seed, or exposing a seed to, a composition of an isolated and biologically pure culture of Bacillus amyloliquefaciens; and, applying to a seed, or exposing a seed to, a composition of at least one LCO. The Bacillus amyloliquefaciens and LCO may also be applied to a furrow in which seeds are planted. One of Bacillus amyloliquefaciens and LCO may be applied to a seed and the other may be applied to a furrow in which the seeds are planted. The seed may be planted.

A fourth aspect provides seeds, wherein the seeds have been exposed to, or have had applied to them, a composition of an isolated and biologically pure culture of Bacillus amyloliquefaciens and at least one LCO.

In a fifth aspect, methods of preparing treated seeds by applying to a seed, or exposing a seed to, a composition of Bacillus amyloliquefaciens and at least one LCO are disclosed. These methods also include applying to a seed, or exposing a seed to, a composition of an isolated and biologically pure culture of Bacillus amyloliquefaciens; and, applying to a seed, or exposing a seed to, a composition of at least one LCO.

In a sixth aspect, plants produced from seeds that have been exposed to, or have had applied to them, a composition of an isolated and biologically pure culture of Bacillus amyloliquefaciens and at least one LCO are disclosed.

In an seventh aspect, kits for improving plant growth and/or improving plant yield that include a composition of an isolated and biologically pure culture of Bacillus amyloliquefaciens and at least one LCO, or separate compositions of Bacillus amyloliquefaciens and of LCO are disclosed. The kits may be used for improving plant growth and/or plant yield. The kit may also include instructions for using the kit. Use of the kits as disclosed results in improved plant growth and/or plant yield as compared to growth and/or yield of plants on which the kits have not been used.

### BRIEF DESCRIPTION OF THE DRAWINGS

In the accompanying figures, which are incorporated in and constitute a part of the specification, compositions and methods for improving plant growth are disclosed. Changes, modifications and deviations from the disclosures illustrated in the figures may be made without departing from the spirit and scope of the invention, as disclosed below.

FIG. 1: Chemical structures of LCOs used in the examples. (A) Chemical structure of an example Nod factor, LCO V (C18:1). (B) Chemical structure of an example Myc factor (LCO IV (C16:0, S)).

FIG. 2: Bacillus amyloliquefaciens increases corn root length. The effect of Bacillus amyloliquefaciens (microbe, filterant, spores) seed treatment on corn root length was measured (in cm) and compared to an untreated control (CHK). Different connecting letters indicate a significant difference in treatments (student's t-test, p≤0.05).

FIG. 3: Bacillus amyloliquefaciens increases corn shoot length. The effect of Bacillus amyloliquefaciens (microbe, filterant, spores) seed treatment on corn shoot length was measured (in cm) and compared to an untreated control (CHK). Different connecting letters indicate a significant difference in treatments (student's t-test, p≤0.05).

FIG. 4: Bacillus amyloliquefaciens and LCO increases corn growth. The effects of Bacillus amyloliquefaciens and LCO seed treatments on corn seedling biomass were measured (in g) and compared to an untreated control (CHK). In addition, the effect of a combined Bacillus amyloliquefaciens and LCO seed treatment was measured. Percentage differences relative to the untreated control are also shown. Different connecting letters indicate a significant difference in treatments (student's t-test, p≤0.05).

### DETAILED DESCRIPTION

### Definitions

The following includes definitions of selected terms and phrases used in the claims. Both singular and plural forms of the terms and phrases fall within the definitions.

By “acaricide” we mean any agent or combination of agents capable of being toxic to an acarid (e.g., mites, ticks), controlling an acarid, killing an acarid, inhibiting the growth of an acarid, and/or inhibiting the reproduction of an acarid. Non-limiting examples of acaricides include permethrin, ivermectin, antibiotic miticides, carbamate miticides, formamidine miticides, organophosphate miticides, diatomaceous earth, dicofol, and lime sulphur.

By “additive effect” we mean that the effect conferred by the combination of two or more factors (e.g. substances, agents, conditions, etc.) is approximately equal to the sum of their separate effects.

By “antioxidant” we mean a molecule that inhibits the oxidation of other molecules, for example, by removing free radical intermediates and terminating free radical-induced chain reactions. Non-limiting examples of antioxidants include flavonoids, polyphenols, vitamin C/ascorbic acid, ethoxyquin, vitamin E, tannins, phytic acid, oxalic acid, glutathione, lactones, lipoic acid, melatonin, uric acid, carotenes, and ubiquinone.

By “applying to” we mean putting or spreading a substance on something else. Application may be effected by any means known in the art. Non-limiting examples of application to a seed or plant include spraying a seed or plant, painting a seed or plant, dipping a seed or plant, submerging a seed or plant, drenching a seed or plant, dripping on a seed or plant, dusting a seed or plant, and coating a seed or plant.

By “combination” we mean two or more substances in proximity to one another and/or used together. In one example, the disclosed compositions of Bacillus amyloliquefaciens and LCOs may be considered a combination. “Combining” refers to an action in placing the Bacillus amyloliquefaciens and LCO in proximity to one another and/or an action in preparation for using the Bacillus amyloliquefaciens and LCO together.

By “fertilizer” we mean a chemical and/or natural substance that can be added to soil to improve plant growth and/or yield of a plant.

By “fungicide” we mean any agent or combination of agents capable of being toxic to a fungus, controlling a fungus, killing a fungus, inhibiting the growth of a fungus, and/or inhibiting the reproduction of a fungus. Non-limiting examples of fungicides include antibiotics, Methyl benzimidazole carbamate (MBC), dicarboximide, demethylation inhibitors (DMI), phenylamide (PA), carboxamide, anilinopyrimidine, quinone outside inhibitor, aromatic hydrocarbons, and host plant defense inducers.

By “furrow” we mean any groove or trough in the ground, in one example, made by a plow.

By “gastropodicide” we mean any agent or combination of agents capable of being toxic to a gastropod, controlling a gastropod, killing a gastropod, inhibiting the growth of a gastropod, and/or inhibiting the reproduction of a gastropod. Non-limiting examples of gastropodicides include copper sulphate, sodium pentachloraphenate, copper pentachlorophenate, the ethanolamine salt of 5,2′-dichloro 4′-nitrosalicylanilide, N-trityl morpholine and tributyltin acetate.

By “germinating” as used in the context of the phrase “germinating the seed”, we mean sprouting of a seedling from a seed in the form of a root, shoot, or other plant structure.

By “herbicide” we mean any agent or combination of agents capable of being toxic to a weed, controlling a weed, killing a weed, inhibiting the growth of a weed, and/or inhibiting the reproduction of a weed. Non-limiting examples of herbicides include ACCase inhibitors, ALS inhibitors, EPSPS inhibitors, synthetic auxins, photosystem I inhibitors, photosystem II inhibitors, HPPD inhibitors, Degree Xtra™, Harness“, Intro™, Lariat™, Micro-Tech™, RoundUp”, RT3™, TripleFlex™, and Warrant™.

By “improve”, “enhance”, “facilitate”, or “promote”, as related to plant growth, we mean that plant growth is generally improved for one or more factors or properties as compared to a standard or control.

By “insecticide” we include the meaning of any agent or combination of agents capable of being toxic to an insect, controlling an insect, killing an insect, inhibiting the growth of an insect, and/or inhibiting the reproduction of an insect. Non-limiting examples of insecticides include organochlorides, organophosphates and carbamates, pyrethroids, neonicotinoids, and ryanoids.

By an “isolated and biologically pure culture” of Bacillus amyloliquefaciens we mean a culture that is substantially biologically pure (i.e., it substantially does not contain other microorganisms), such as, at least 90% pure, preferably at least 95% pure, more preferably 97% pure, yet more preferably at least 99% pure, most preferably 100% pure.

By “kit,” we mean a set or collection of two or more things, generally for a purpose. The two or more things that are part of a kit may be said to be “packaged” into or as a kit.

By “lipochitooligosaccharide” (LCO) (also known as lipo-chitin oligosaccharides) we mean those LCOs obtained or purified from bacterial or fungal species (for example, Nod factors and Myc factors), as well as synthetic LCO compounds, for example those described in WO 2005/063784 and WO 2008/071674, chemically synthesized LCO compounds, for example those described in WO 2007/117500, and recombinant LCO's produced through genetic engineering. The basic, naturally occurring LCO structure may contain modifications or substitutions found in naturally occurring LCO's, for example, those described in Spaink, 2000. Crit. Rev. Plant Sci., 54:257 288 and D'Haeze, et al, 2002. Glycobiology, 12:79R-105R.

By “microorganism” or “microbe” we mean microscopic organisms, generally too small to be viewed by the naked eye. Example microorganisms include bacteria, archaea, protozoa, and some fungi and algae. Microbe generally includes all forms/stages of an organism. In one example, a named microbe that can sporulate, includes both the vegetative form and spore form of the microbe, unless indicated otherwise.

By “more than additive effect” we mean that the effect conferred by the combination of two or more factors (e.g. substances, agents, conditions, etc.) is greater than the sum of their separate effects.

By “Myc factor” we mean LCOs produced by Mycorrhizal fungi.

By “nematicide” we mean any agent or combination of agents capable of being toxic to a nematode, controlling a nematode, killing a nematode, inhibiting the growth of a nematode, and/or inhibiting the reproduction of a nematode. Non-limiting examples of nematicides include carbofuran, aldoxycarb, dazomet methyl bromide, carbamates, and aldicarb.

By “Nod factor” we mean LCOs produced by bacteria.

By “pest” we mean any organism or virus, (e.g., invertebrates, microorganisms, viruses, etc.) which negatively affects plants. This includes organisms or viruses that spread disease and/or damage the host and/or compete for host nutrients. In addition, plant pests are organisms or viruses known to associate with plants and which, as a result of that association, cause a detrimental effect on the plant's health and vigor. Plant pests include, but are not limited to, invasive plants (e.g., weeds), fungi, bacteria, insects (e.g., white flies, thrips, weevils, etc.), arachnids (e.g., mites, ticks, spiders, etc.), nematodes (e.g., root-knot nematode, soybean cyst nematode, etc.), viruses (e.g., tobacco mosaic virus (TMV), tomato spotted wilt virus (TSWV), cauliflower mosaic virus (CaMV), etc.), gastropods (e.g., slugs, snails, etc.), and the like.

By “pesticide” we mean an agent or a combination of agents that is capable of being toxic to a pest, killing a pest, controlling a pest, inhibiting the growth of a pest, and/or inhibiting the reproduction of a pest. Non-limiting examples of pesticides include fungicides, herbicides, insecticides, acaricides, nematicides, rodenticides, virucides, gastropodicides, etc.

By “plant” we mean a living organism that typically grows in soil, absorbing water and inorganic substances through roots and synthesizing nutrients by photosynthesis. Plant includes all plants and plant populations, for example, desired and undesired wild plants or crop plants (including naturally occurring crop plants). Typical plants may include trees, shrubs, herbs, grasses, ferns, mosses, flowers, fruit, vegetables, houseplants and others. Plants may be monocotyledonous or dicotyledonous. A plant may include the entirety of a plant or may include one or more forms, parts and/or organs of a plant, above or below ground. Plant includes all plant forms, parts and/or organs which may include, for example, shoots, leaves, flowers, roots, needles, stalks, stems, flowers, fruit bodies, fruits, seeds, roots, tubers, rhizomes, and the like. Plants may also include harvested material and vegetative and generative propagation material (e.g., cuttings, tubers, rhizomes, off-shoots and seeds, etc.). One example plant is corn or maize.

Use of the word “plant” as a verb (e.g., “planting”), with reference to a planted seed or seedling, or planting a seed or seedling, refers to placing or locating a seed or seedling in an environment (e.g., soil) where the seed or seedling can grow.

By “plant growth” we mean all or part of the process that begins with a plant seed and continues to a mature plant. Generally, as a plant grows and/or matures from a seed planted in soil, the seed germinates, the plant emerges from the soil, and roots, stems and leaves form. Generally, as a plant grows, it will increase in size and mass. Plant growth may be determined by observing one or more aspects of a plant. For example, growth rate, amount of yield, root number, root length, root mass, root yield, shoot length, shoot mass, shoot yield, leaf area, plant stand, plant vigor, dry weight of roots, dry weight of shoots, increased root/shoot volume, increased plant stand, increased plant vigor, total plant biomass, increased fruit number, increased bolls, increased seed number or size, increase in germination frequency, decrease in time for germination to occur, or any of a number of other factors, individually or collectively, may be properties that may be observed and may correlate with plant growth.

By “plant growth-promoting bacteria” (PGPB) we mean any microorganism which facilitates plant growth by, for example, making nutrients available to a plant (e.g. an organism that makes phosphate available to a plant). We also include the meaning of any microorganism which facilitates plant growth by controlling a pest organism. For example, bacteria which produce compounds which kill or inhibit the growth of other bacteria or other microorganisms.

By “plant hormone” (also known as phytohormone or plant growth substance) we mean any plant-produced substance or chemical which can regulate germination, growth, metabolism, or other physiological activities of a plant. Non-limiting examples of plant hormones include auxins, cytokinins, gibberellins, ethylene, abscisic acid, brassinosteroids, salicylic acid and its derivatives, jasmonates and its derivatives, plant peptide hormones, polyamines, nitric oxide, and strigolactones.

By “rodenticide” we mean any agent or combination of agents capable of being toxic to a rodent, controlling a rodent, killing a rodent, inhibiting the growth of a rodent, and/or inhibiting the reproduction of a rodent. Non-limiting examples of rodenticides include anticoagulants, metal phosphides, hypercalcemia-inducing compounds (e.g. calciferols), arsenic trioxide, barium carbonate, chloralose, sodium fluoroacetate, thallium chloride, and nitrophenols.

By “soilless medium” we mean a medium, generally for plants, that does not contain soil. Soilless media may include, but is not limited to, hydroculture (including hydroponics), aeroponics, and fogponics.

By “supplying to”, “contacting with”, or “exposing to” we mean putting, placing, or applying the compositions of the invention at a site, other than on the seed, that is in close enough proximity to the seed such that the compositions are capable of improving or facilitating plant growth directly and/or indirectly. For example, when specifically used in the context of a composition comprising bacteria, the terms include the meaning of applying, putting, or placing the composition in close enough proximity that the bacteria, or substances produced by the bacteria, are capable of improving or facilitating plant growth, directly and/or indirectly. In one example, applying the compositions disclosed herein to a furrow in which a seed is planted may be an example of supplying the composition.

By “virucide” we mean any agent or combination of agents capable of being toxic to a virus, controlling a virus, killing a virus, and/or inhibiting the reproduction of a virus. Non-limiting examples of virucides include cyanovirin-N, griffithsin, scytovirin, Virkon, NVC-422, zidovudine, Zonrox, interferon, and Lysol.

Bacteria

In one example, the bacteria used in the compositions and methods disclosed here may be plant growth-promoting bacteria (PGPB). PGPB are naturally (i.e. in the wild) associated with many, if not all, plant species and are present in many environments (Hayat et al, 2010. Ann. Microbiol., 60:579-598). Bacteria of that classification are generally divided into two broad groups: extracellular, free-living bacteria which usually inhabit an area called the rhizosphere; and intracellular bacteria, which are usually nitrogen-fixing bacteria. In one example, the bacteria used in the compositions disclosed here may not be PGPB.

PGPBs include, but are not limited to, plant growth-promoting rhizobacteria (for example, Rhizobium sp., Bradyrhizobium sp., Sinorhizobium sp, Azorhizobium sp., etc.), Pseudomonas sp., Bacillus sp., Enterobacter sp., and Atherobacter sp. Many of those bacteria colonize the rhizosphere, which is an area encompassing the roots, root surfaces, and the closely adhering soil interface (McNear, 2013. Nature Education Knowledge, 4(3):1). PGPB and other microorganisms living in the rhizosphere, for example, certain fungal species, may promote growth through a variety of different direct or indirect mechanisms. For example, plant growth promotion can be shown to work directly on the plant through the release of plant growth-stimulating compounds (for example, molecules known as lipochitooligosaccharides) and/or improvement in mineral uptake (e.g. siderophore release increasing iron availability; solubilization of phosphate for plant uptake). Plant growth promotion can also occur indirectly by control of pathogens (biocontrol) via synthesis of antibiotics or secondary metabolite-mediated induced systemic resistance (ISR) (van Loon, et al., 1998. Annual Review of Phytopathology, 36:453-483; van Loon et al, 2007. Eur. J. Plant Pathol., 119:243-254). Often the relationship between a PGPB and a host plant is symbiotic in nature. PGPBs have been shown to increase plant growth and productivity for a number of commercially important crops including rice (Ashrafuzzaman et al, 2009. Afr. J. Biotech., 8(7):1247-1252), wheat (Khalid et al, 2004. J. Appl. Microbial., 96(3): 473-480), cucumber (Maleki et al, 2010. AJCS, 4(9):676-683), corn (Sandhya et al, 2010. Plant Growth Regulation, 62(1):21-30), cotton (Anjum et al, 2007. J. Agri. Res., 45:135-143), black pepper (Datta et al, 2011. AJCS, 5(5):531-536), and banana (Mia et al, 2010. AJCS, 4(2):85-90).

Generally, herein, a bacterium that, when supplied to a plant facilitates growth of the plant is considered a PGPB. In one example, PGPB may have biocontrol activity. Biocontrol activity may include fungicidal, gastropodicidal, herbicidal, insecticidal, nematicidal, pesticidal, rodenticidal, virucidal, and the like. In one example, PGPB may have biocontrol-independent activity (e.g., activity that increases nutrient availability to plants).

In one example, the bacteria used herein are from the genus Bacillus. The Bacillus bacterium may be a Bacillus amyloliquefaciens bacterium. The Bacillus amyloliquefaciens may be subsp. plantarum (Bacillus amyloliquefaciens subsp. plantarum is sometimes now called Bacillus methylotrophicus) or subsp. amyloliquefaciens. In one example, the Bacillus amyloliquefaciens may be strain SB3281 (ATCC # PTA-7542), which is subsp. plantarum. These bacteria generally may have biocontrol activity, biocontrol-independent activity, or both biocontrol and biocontrol-independent activity. In one example, Bacillus amyloliquefaciens strain SB3281 includes variants of strain SB3281, mutants of strain SB3281, progeny of strain SB3281, and the like.

In addition to the Bacillus amyloliquefaciens strain SB3281, deposited as PTA-7542, other strains of Bacillus used herein may include those deposited as PTA-7541, PTA-7543, PTA-7544, PTA-7545, PTA-7546, PTA-7547, PTA-7548, PTA-7549, PTA-7550, PTA-7789, PTA-7790, PTA-7791, PTA-7792, and PTA-7793 (see U.S. Pat. Nos. 8,383,097, 8,628,765, and 9,193,940). Mixtures of two or more of these or other Bacillus strains may be used.

The bacteria used in the disclosed compositions and methods may be isolated and/or present in a biologically pure culture.

In one example, the bacteria described herein may be used in the compositions and methods disclosed herein. In one example, the bacteria may produce spores, and the spores may be used in the compositions and methods disclosed herein. Methods for producing spores are well known in the art. In one example, cell-free media in which the bacteria have been grown (e.g., bacteria cultured in the media, cells removed by centrifugation or filtration) may be used in the compositions and methods disclosed herein.

Generally, the amount or number of Bacillus amyloliquefaciens used in the disclosed compositions and methods is an amount that, when used in combination with an amount of LCO, improves plant growth as compared to either the Bacillus amyloliquefaciens alone or LCO alone. Generally, the effect of the combination of Bacillus amyloliquefaciens and LCO is more than additive as compared to the sum of the effects of Bacillus amyloliquefaciens alone and LCO alone. In one example, the effect of the combination may be additive of the separate effects of Bacillus amyloliquefaciens alone and LCO alone. In one example, the number of Bacillus amyloliquefaciens (e.g., colony forming units) applied to plants may be at least 10², 10³, 10⁴, 10⁵, 10⁶, 10⁷, 10⁸, or 10⁹. These numbers of bacteria may be applied or exposed to a seed or plant.

### LCOs

The bacteria may be combined with and/or used together with lipochitooligosaccharides (LCOs) in the compositions and methods disclosed herein. In one example the LCOs may be Nod factors or Myc factors. Nod factors, also known as symbiotic Nod signals, consist of an oligosaccharide backbone of β 1,4 linked N acetyl D glucosamine (“GlcNAc”) residues with an N linked fatty acyl chain condensed at the non-reducing end. Nod factors differ in the number of GlcNAc residues in the backbone, in the length and degree of saturation of the fatty acyl chain, and in the substitutions of reducing and non-reducing sugar residues. For example, see Denarie et al, 1996. Ann. Rev. Biochem., 65:503-35; Hamel et al, 2010. Planta, 232:787-806; and, Prome et al, 1998. Pure & Appl. Chem., 70(1):55-60. Such factors may be isolated and/or purified from bacteria, for example, Rhizobia, e.g. Rhizobium sp., Bradyrhizobium sp., Sinorhizobium sp., and Azorhizobium sp.

Nod factors may promote the initiation of the formation of nodules in legumes, and a symbiotic relationship is generally formed when the Nod-producing bacteria are taken up by the legume. Once that relationship is formed the Nod-producing bacteria fix atmospheric nitrogen which the legume can use for growth.

Myc factors are similar in structure to Nod factors and are often considered to be ancestors of the more recent Nod factors. Myc factors have been shown to promote a type of root endosymbiosis between fungi and plants called arbuscular mycorrhiza (AM). This relationship is the most common terrestrial plant symbiosis and is associated with improved plant uptake of water and mineral nutrients (Maillet et al, 2011. Nature, 469:58-64). Myc factors may be isolated and/or purified from mycorrhizal fungi, for example, fungi of the group Glomerocycota, e.g., Glomus intraradicus.

In some examples, the compositions and methods disclosed herein may comprise one or more LCOs represented by formula I:

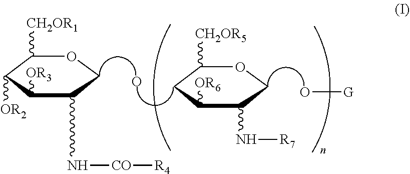

in which G is a hexosamine which can be substituted, for example, by an acetyl group on the nitrogen, or a sulfate group, an acetyl group and/or an ether group on an oxygen; R₁, R₂, R₃, R₅, R₆ and R₇, which may be identical or different, represent H, CH₃ CO—, CₓH_{y} CO— where x is an integer between 0 and 17 and y is an integer between 1 and 35, or any other acyl group such as, for example, a carbamoyl; R₄ represents a saturated or mono-, di- or tri-unsaturated aliphatic chain containing at least 12 carbon atoms; and n is an integer between 1 and 4.

LCOs may be obtained (i.e., isolated and/or purified) from bacteria and fungi. The structural characteristics of naturally occurring LCOs vary depending on the species/strain from which they are obtained and are thought to be the primary determinant of host specificity in the symbiotic nodulation/mycorrhization relationships that exist between plants and naturally occurring soil bacteria/fungi. See, e.g., Diaz et al., MOL. PLANT-MICROBE INTERACTIONS 13:268 (2000); Hungria et al., SOIL BIOL. BIOCHEM. 29:819 (1997). Examples of symbiotic relationships between bacteria and plants include S. meliloti with alfalfa and sweet clover, R. leguminosarum biovar viciae with peas and lentils, R. leguminosarum biovar phaseoli with beans, Bradyrhizobium japonicum with soybeans and R. leguminosarum biovar trifolii with red clover.

As will be understood by those skilled in the art, a given bacterial/fungal strain may produce multiple LCOs. For example, strains of S. meliloti produce LCOs represented by formula II:

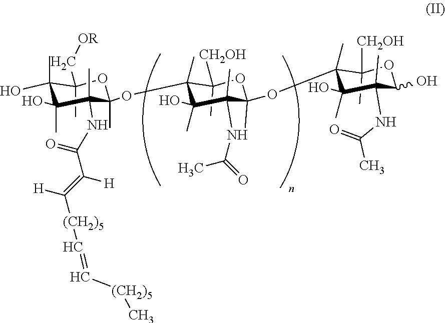

in which R represents H or CH₃ CO— and n is equal to 2 or 3. See, e.g., U.S. Pat. No. 5,549,718. A number of Bradyrhizobium japonicum-derived LCOs have also been described, including BjNod-V (C_{18:1}), BjNod-V (Ac, C_{18:1}), BjNod-V (C_{16:1}) and BjNod-V (Ac, C_{16:0}) (with “V” indicating the presence of five N-acetylglucosamines, “Ac” an acetylation, the number following the “C” indicating the number of carbons in the fatty acid side chain and the number following the “:” indicating the number of double bonds). See, e.g., U.S. Pat. Nos. 5,175,149 and 5,321,011. Additional LCOs obtained from bacterial strains include NodRM, NodRM-1, NodRM-3. When acetylated (the R=CH₃ CO—), they become AcNodRM-1 and AcNodRM-3, respectively (U.S. Pat. No. 5,545,718).

Representative fungal-derived LCOs and non-naturally occurring derivatives thereof are represented by formula III:

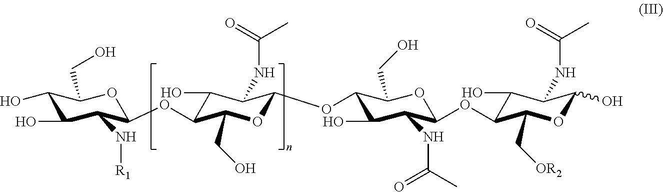

in which n=1 or 2; R₁ represents C16, C16:0, C16:1, C16:2, C18:0, C18:1Δ9Z or C18:1Δ11Z; and R₂ represents hydrogen or SO₃H.

LCOs included in the compositions and methods disclosed here may be obtained from any suitable source.

In some examples, the LCO is obtained (i.e., isolated and/or purified) from a naturally occurring or non-naturally occurring bacterial strain. For example, in some embodiments, inoculant compositions of the present invention comprise one or more LCOs obtained from a naturally occurring or genetically engineered strain of Azorhizobium, Bradyrhizobium (e.g., B. japonicum), Mesorhizobium, Rhizobium (e.g., R. leguminosarum), or Sinorhizobium (e.g., S. meliloti). These LCOs may be called Nod factors.

In some examples, the LCO is obtained (i.e., isolated and/or purified) from a naturally occurring or non-naturally occurring mycorrhizal fungus. For example, in some embodiments, inoculant compositions of the present invention comprise one or more LCOs obtained from a naturally occurring or genetically engineered strain of Glomerocycota (e.g., Glomus intraradicus). See, e.g., WO 2010/049751 (in which the LCOs are referred to as “Myc factors”).

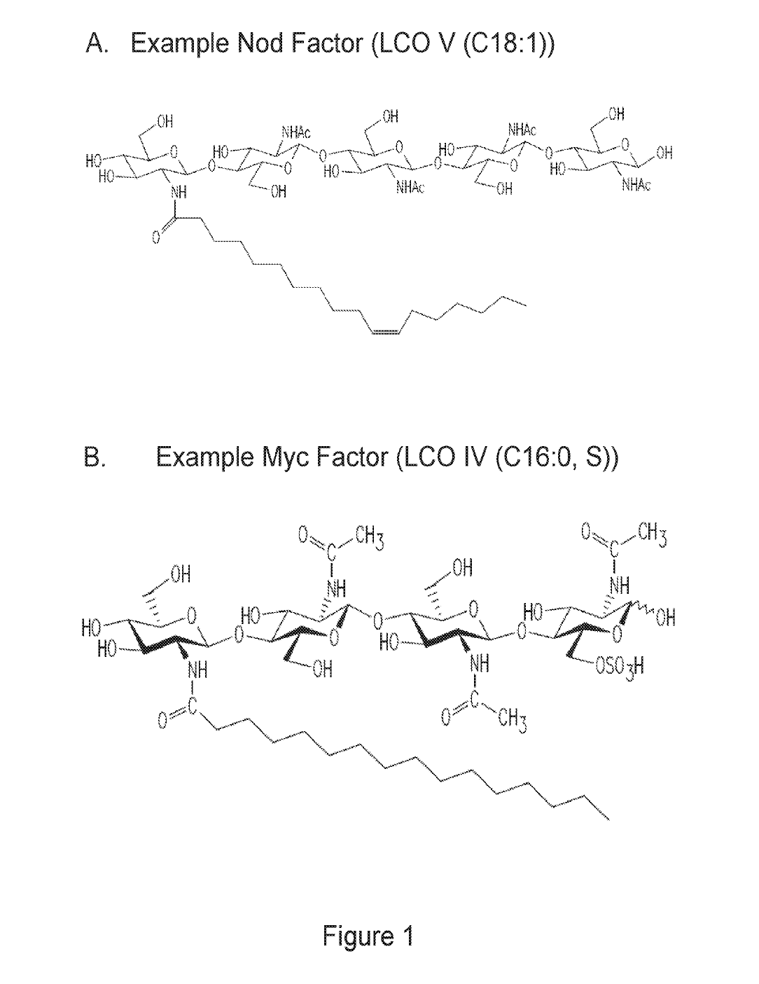

In some examples, the LCO is synthetic. For example, in some examples, inoculant compositions disclosed here comprise one or more of the synthetic LCOs described in WO 2005/063784, WO 2007/117500 and/or WO 2008/071674. In some examples, the synthetic LCO has the basic structure of a naturally occurring LCO but contains one or more modifications or substitutions, such as those described in Spaink, CRIT. REV. PLANT SCI. 54:257 (2000) and D'Haeze, supra. Precursors for the construction of LCOs (e.g., COs, which are themselves useful as plant signal molecules) may be synthesized by genetically engineered organisms. See, e.g., Samain et al., CARBOHYDRATE RES. 302:35 (1997); Cottaz et al., METH. ENG. 7(4):311 (2005); and Samain et al., J. BIOTECHNOL. 72:33 (1999) (e.g., FIG. 1 therein, which shows structures of COs that can be made recombinantly in E. coli harboring different combinations of genes nodBCHL).

Further examples of LCOs (and derivatives thereof) that may be useful in compositions and methods of the present invention are provided below as formula IV:

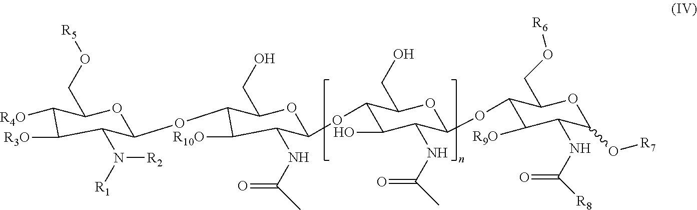

in which R₁ represents C14:0, 30H—C14:0, iso-C15:0, C16:0, 3-OH—C16:0, iso-C15:0, C16:1, C16:2, C16:3, iso-C17:0, iso-C17:1, C18:0, 30H—C18:0, C18:0/3-OH, C18:1, OH—C18:1, C18:2, C18:3, C18:4, C19:1 carbamoyl, C20:0, C20:1, 3-OH—C20:1, C20:1/3-OH, C20:2, C20:3, C22:1 and C18-26(ω-1)-OH (which according to D'Haeze et al., Glycobiology 12:79R-105R (2002), includes C18, C20, C22, C24 and C26 hydroxylated species and C16:149, C16:2 (Δ2,9) and C16:3 (Δ2,4,9)); R₂ represents hydrogen or methyl; R₃ represents hydrogen, acetyl or carbamoyl; R₄ represents hydrogen, acetyl or carbamoyl; R₅ represents hydrogen, acetyl or carbamoyl; R₆ represents hydrogen, arabinosyl, fucosyl, acetyl, SO₃H, sulfate ester, 3-0-S-2-0-MeFuc, 2-0-MeFuc and 4-0-AcFuc; R₇ represents hydrogen, mannosyl or glycerol; R₈ represents hydrogen, methyl, or —CH₂OH; R₉ represents hydrogen, arabinosyl, or fucosyl; R₁₀ represents hydrogen, acetyl or fucosyl; and n represents 0, 1, 2 or 3. Naturally occurring LCOs embraced by this structure are described in D'Haeze et al., supra.

With reference to structure IV, example LCOs may contain:

At R1, fatty acid chains containing—C18:1, Δ11 (vaccenic acid); C16:1, Δ9 (oleic acid); C16:1, Δ11 (palmitoleic acid), C16:0 (palmitic acid), C18:1, Δ9 (oleic acid); C18:1, Δ11 (cis-vaccenic acid) and the like; at R2, H; at R3, H; at R4, H; at R5, an H or acetyl group; at R6, an H, fucose or S; at R7, H; at R8, methyl; at R9, H; at R10, H; and n=1 or 2.

In one example, R1 is C18:1, Δ11 (vaccenic acid); R2-R7 are H; R8 is methyl; R9-10 are H; n is 2.

In one example, R1 is C16:0 (palmitic acid); R2-R5 are H; R6 is sulfate; R7 is H; R8 is methyl; R9-R10 are H; n is 1.

Further examples of LCOs (and derivatives thereof) that may be useful in compositions and methods disclosed herein are provided below as structures V-XXXIII:

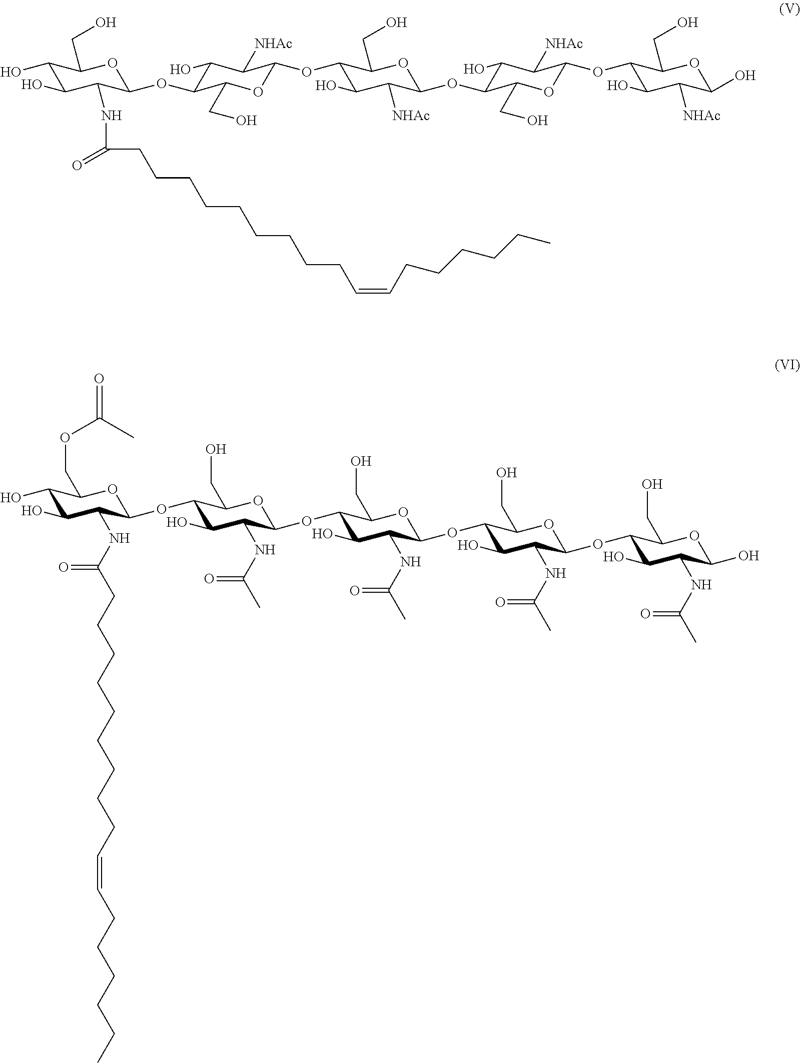

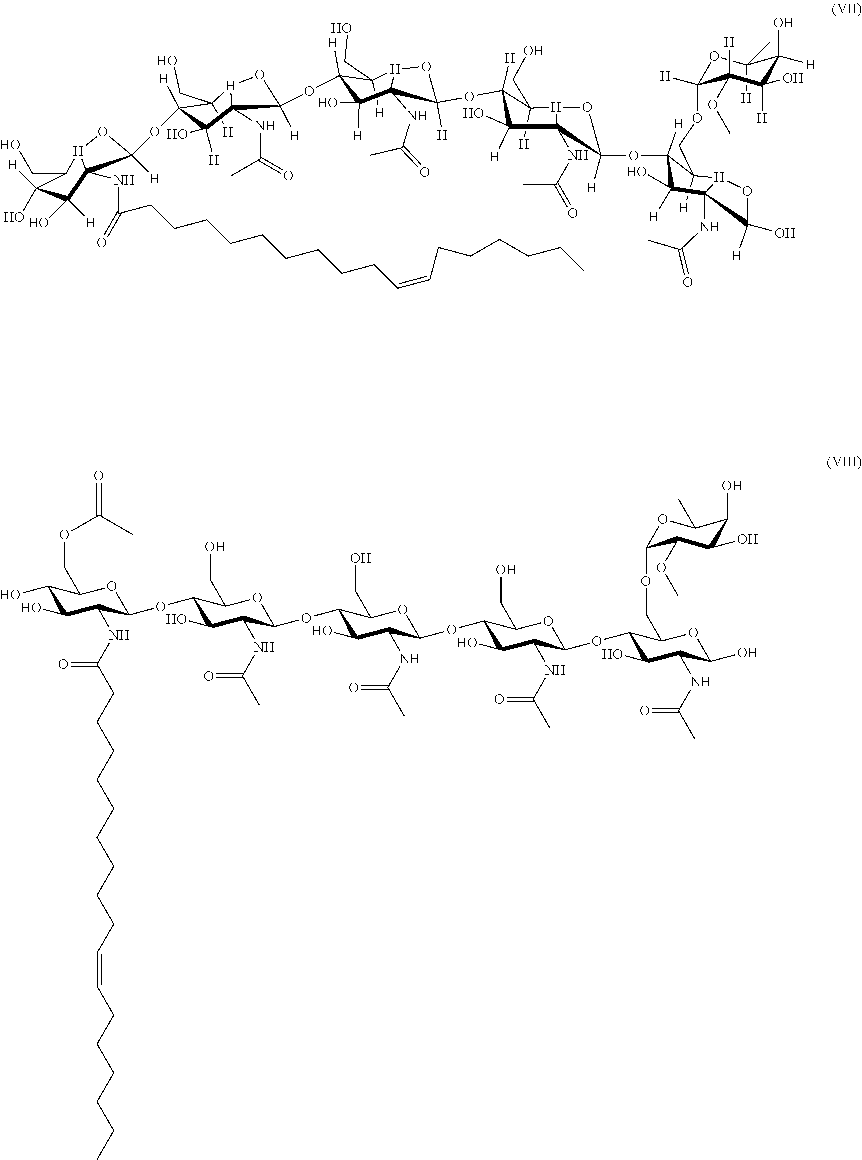

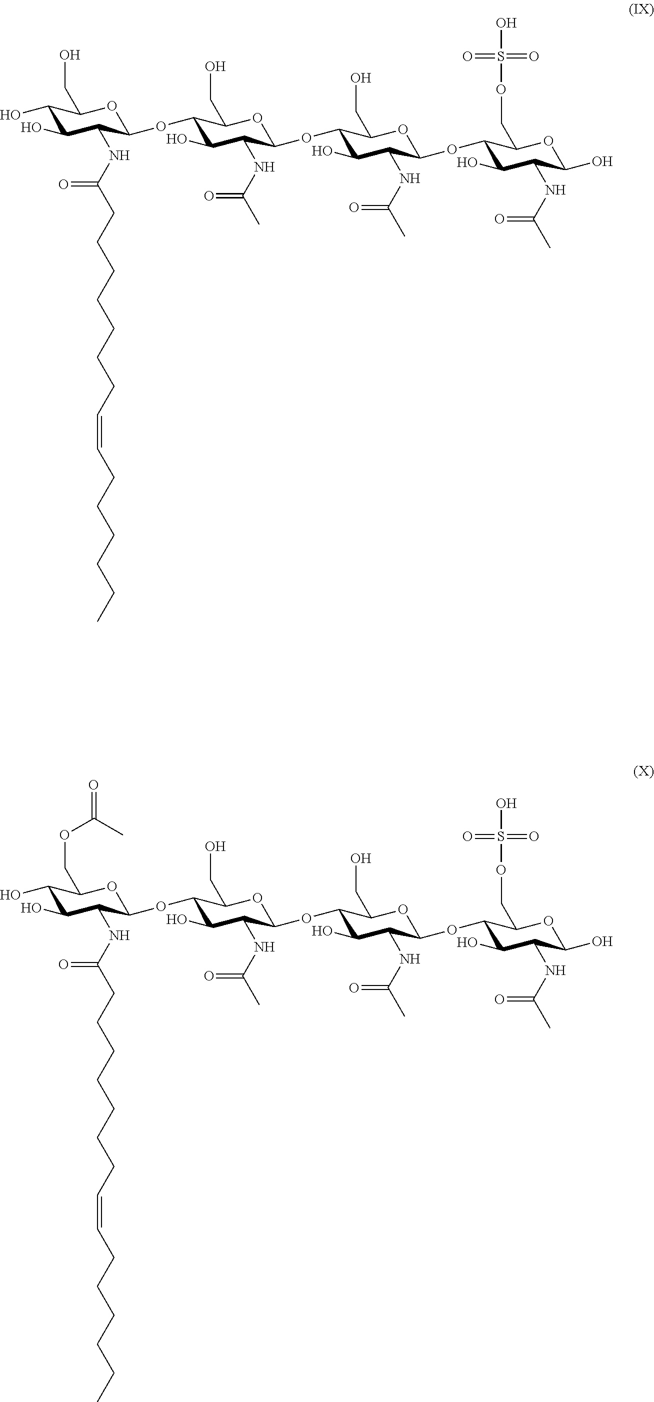

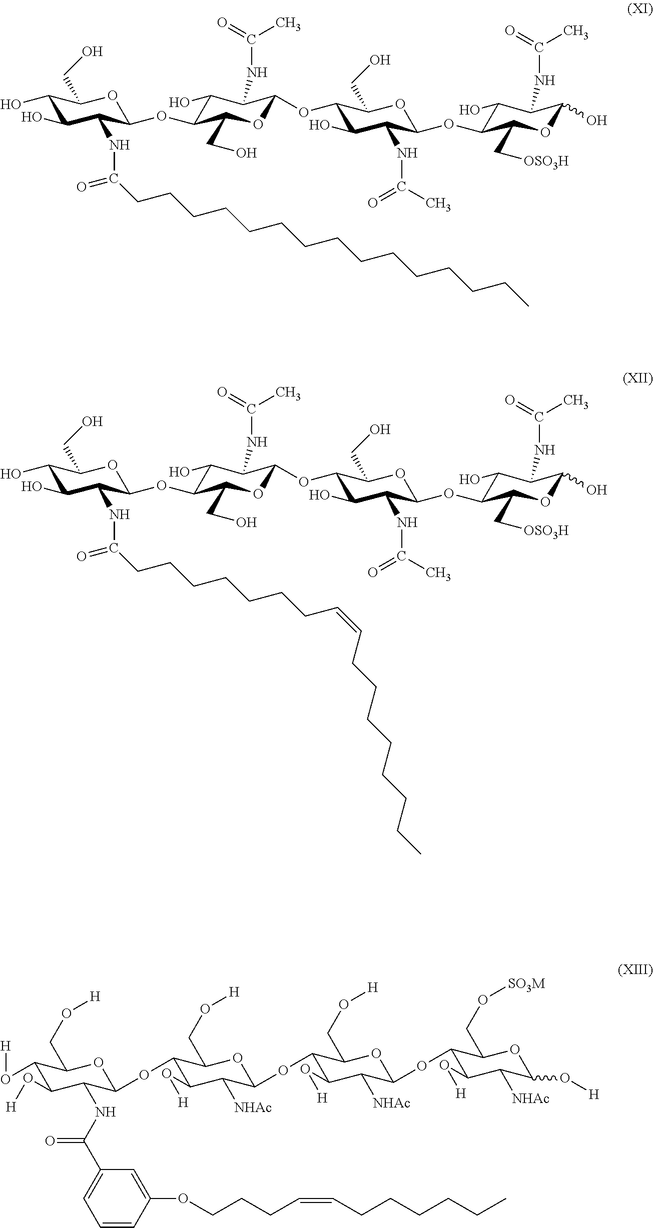

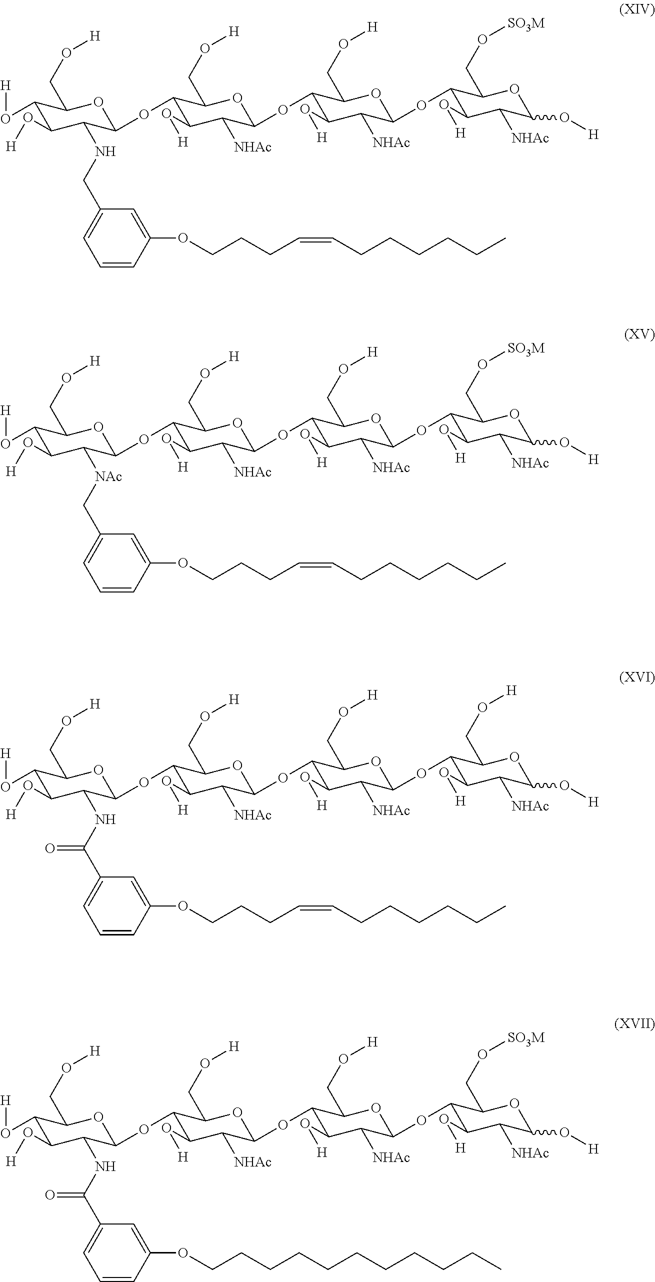

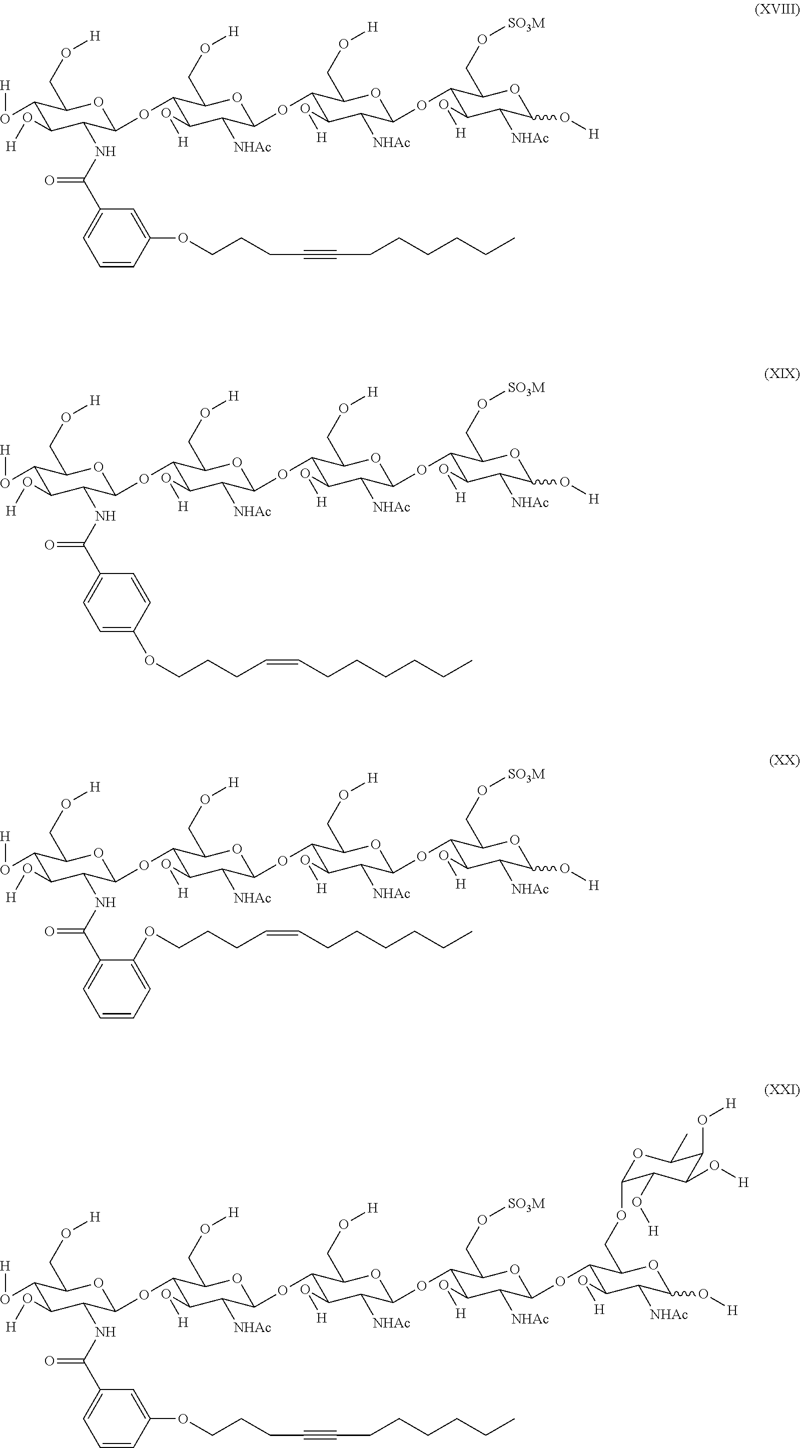

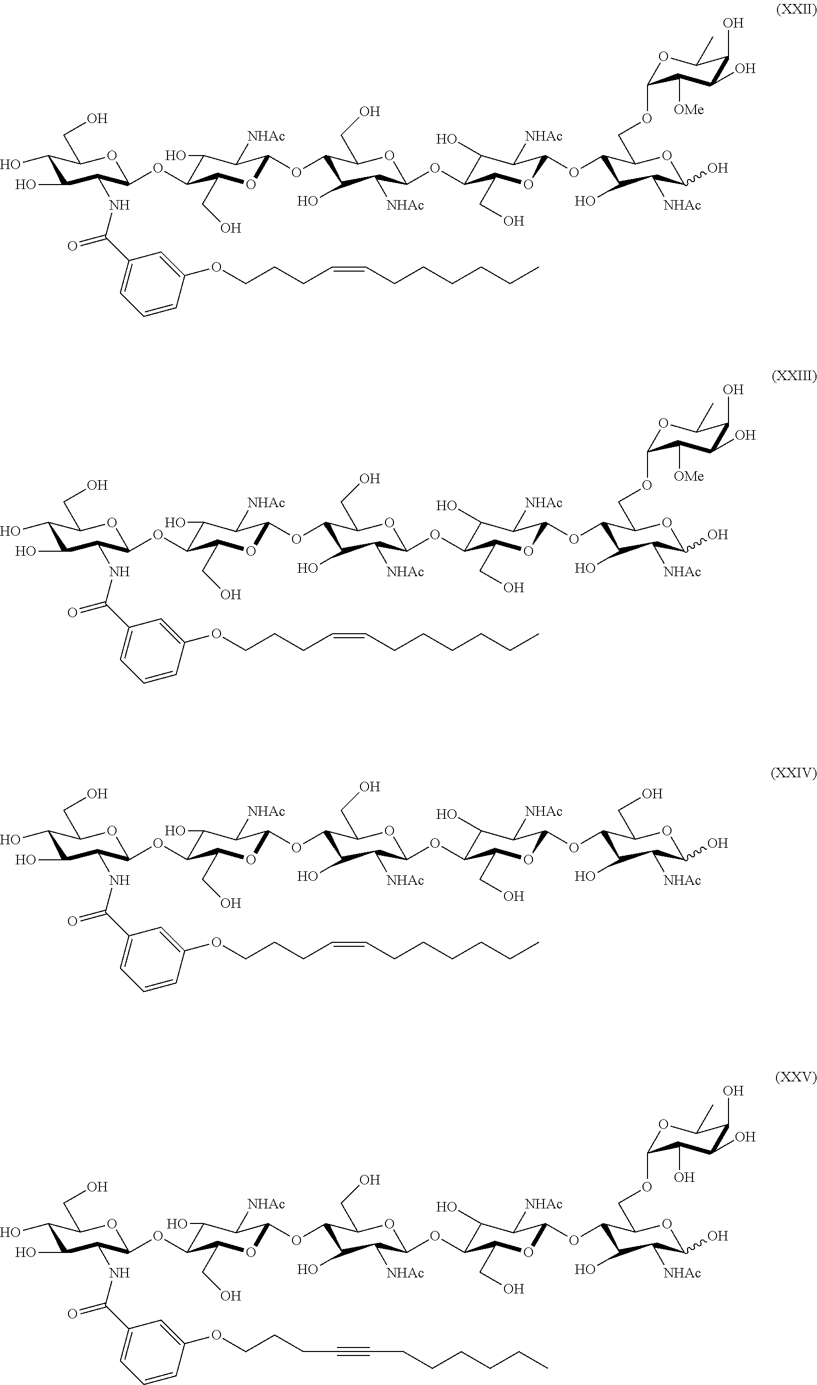

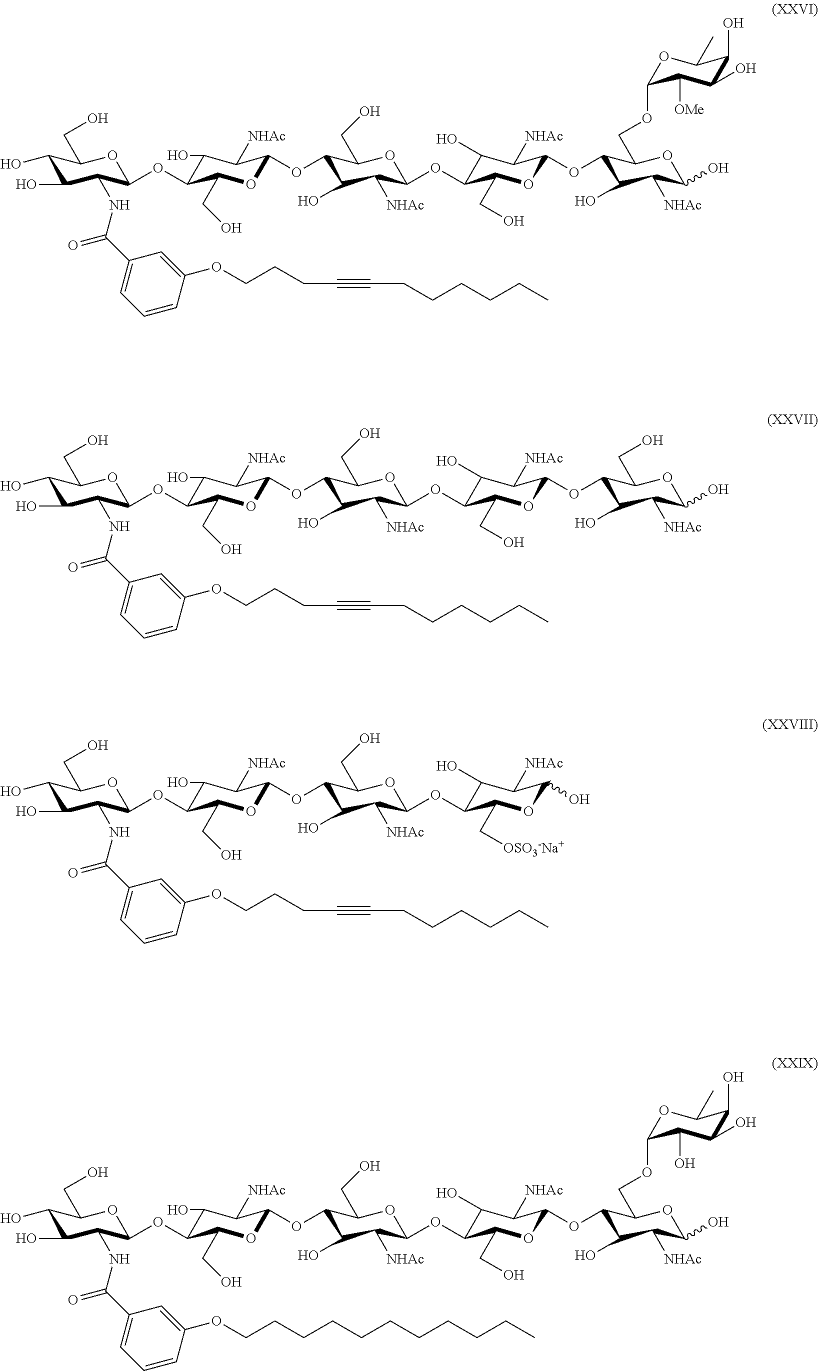

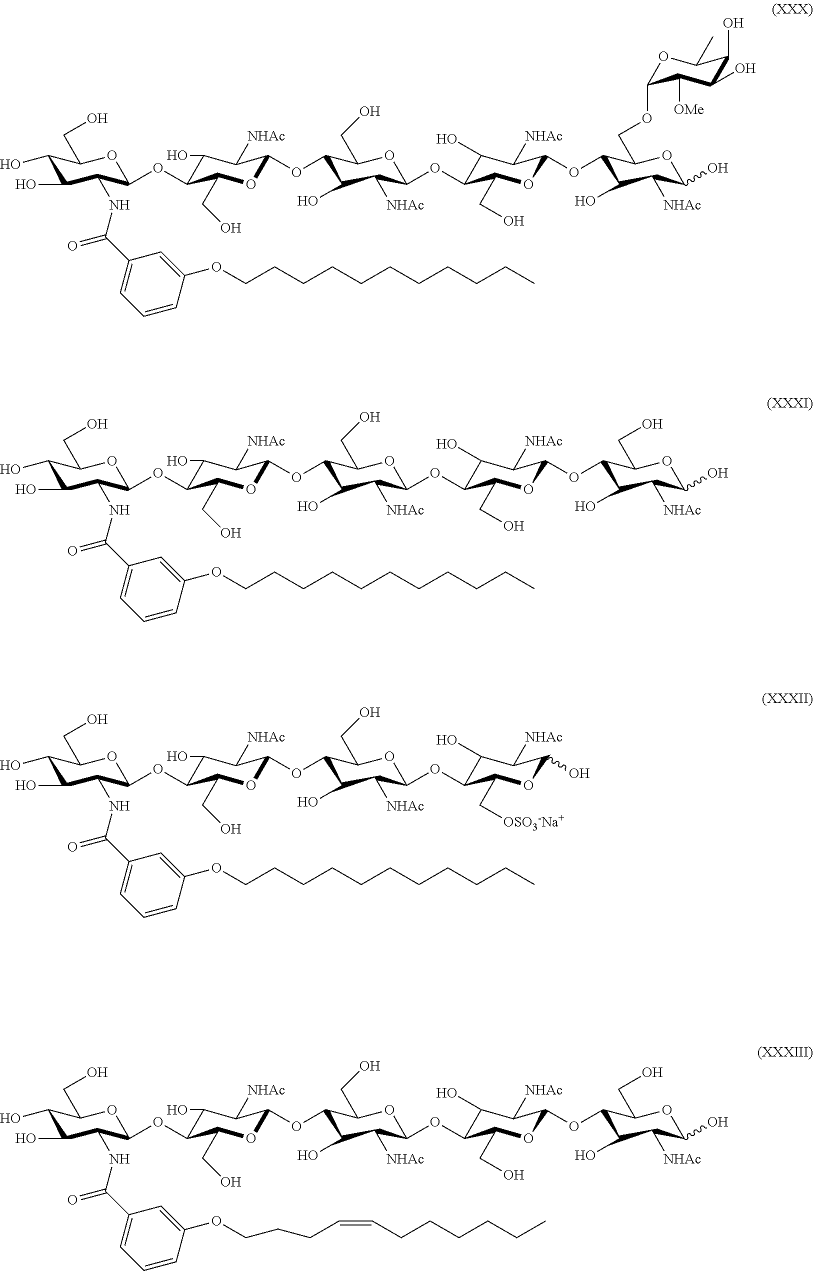

It is to be understood that, although the naturally occurring symbiotic relationships between bacteria/fungi and plants are governed by the structural characteristics of the LCOs involved, compositions and methods disclosed herein are not so limited. A given LCO may be utilized in compositions and methods disclosed herein to enhance the growth and/or yield of a plant with which it is not naturally compatible (i.e., it can be used to enhance the growth/yield of a plant even if it is not capable of inducing nodulation in that plant). For example, inoculant compositions comprising an LCO obtained from a naturally occurring strain of S. meliloti may be used to enhance the growth/yield of soybean plants (as evidenced by enhanced biomass, bushels per acre, chlorophyll content, drought tolerance, height, leaf length, leaf mass, leaf number, leaf surface area, leaf volume, nutrient uptake (e.g., nitrogen and/or phosphorous uptake), nutritional content, PB, PYREC, rate of photosynthesis, root length, root mass, root nodulation, root number, root surface area, root volume, seed germination, seedling emergence, spread and survival rate, YPP, YRED and/or YSMP), compared to plants harvested from untreated seed.

LCOs (and derivatives thereof) may be utilized in various forms of purity and may be used alone or in the form of a culture of LCO-producing bacteria or fungi. For example, OPTIMIZE® contains a culture of B. japonicum that produces an LCO (LCO-V(C18:1, MeFuc)). Methods to provide substantially pure LCOs include removing the microbial cells from a mixture of LCOs and the microbe, or continuing to isolate and purify the LCO molecules through LCO solvent phase separation followed by HPLC chromatography as described, for example, in U.S. Pat. No. 5,549,718. Purification can be enhanced by repeated HPLC and the purified LCO molecules can be freeze-dried for long-term storage. In some examples, the LCO(s) included in the compositions and methods disclosed herein are at least 70, 75, 80, 85, 90, 91, 92, 93, 94, 95, 96, 97, 98, 99, 99.5% or more pure.

It is to be understood that compositions and methods disclosed herein may comprise analogues, derivatives, hydrates, isomers, salts and/or solvates of LCOs.

Thus, in some examples, inoculant compositions may comprise one, two, three, four, five, six, seven, eight, nine, ten, or more LCOs represented by one or more of formulas I—IV and/or structures V-XXXIII and/or one, two, three, four, five, six, seven, eight, nine, ten, or more analogues, derivatives, hydrates, isomers, salts and/or solvates of LCOs represented by one or more of formulas I-IV and/or structures V-XXXIII

LCOs may be incorporated into compositions disclosed herein in any suitable amount(s)/concentration(s).

In some examples, the compositions disclosed herein comprise about 1×10⁻²⁰ M to about 1×10⁻¹ M LCO. For example, compositions may comprise about 1×10⁻²⁰ M, 1×10⁻¹⁹ M, 1×10⁻¹⁸ M, 1×10⁻¹⁷ M, 1×10⁻¹⁶ M, 1×10⁻¹⁵ M, 1×10⁻¹⁴ M, 1×10⁻¹³ M, 1×10⁻¹² M, 1×10⁻¹¹ M, 1×10⁻¹⁰ M, 1×10⁻⁹ M, 1×10⁻⁸ M, 1×10⁻⁷ M, 1×10⁻⁶ M, 1×10⁻⁵ M, 1×10⁻⁴ M, 1×10⁻³ M, 1×10⁻² M, 1×10⁻¹ M of one or more LCOs. In some examples, the LCO concentration is 1×10⁻¹⁴ M to 1×10⁻⁵ M, 1×10⁻¹² M to 1×10⁻⁶ M, or 1×10⁻¹⁰ M to 1×10⁻⁷ M. In some examples, the LCO concentration is 1×10⁻¹⁴ M to 1×10⁻⁵ M, 1×10⁻¹² M to 1×10⁻⁶ M, or 1×10⁻¹⁰ M to 1×10⁻⁷ M.

In some examples, the compositions comprise an LCO at a concentration of at least 1.0 nM. In some examples, the compositions comprise an LCO at a concentration of at least 0.1 nM, 0.2 nM, 0.3 nM, 0.4 nM, 0.5 nM, 0.6 nM, 0.7 nM, 0.8 nM, 0.9 nM, 1.0 nM, 2.0 mM, 3.0 mM, 4.0 mM, 5.0 mM, 6.0 mM, 7.0 mM, 8.0 mM, 9.0 mM, 10.0 mM, 11.0 mM, 12.0 mM, 13.0 mM, 14.0 mM, 15.0 mM, 16.0 mM, 17.0 mM, 18.0 mM, 19.0 mM, 20 mM, 25 mM, 30 mM, 35 mM, 40 mM, 45 mM, 5 OmM, 55 mM, 60 mM, 65 mM, 70 mM, 75 mM, 80 mM, 85 mM, 90 mM, 95 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 525 mM, 550 nM, 575 nM, 600 nM, 625 nM, 650 nM, 675 nM, 700 nM, 725 nM, 750 nM, 775 nM, 800 nM, 825 nM, 850 nM, 875 nM, 900 nM, 925 nM, 950 nM, 975 nM, 1.0 2.0 5.0 or 10 μM.

In certain examples, the disclosed compositions comprise an LCO at a concentration of between 0.1 nM and 10 0.1 nM and 10 nM, 0.1 nM and 20 nM, 0.1 nM and 50 nM, 0.1 nM and 100 nM, 0.2 nM and 10 0.3 nM and 10 0.4 nM and 10 0.5 nM and 10 0.6 nM and 10 0.7 nM and 10 0.8 nM and 10 0.9 nM and 10 μM, 1.0 nM and 250 nM, 2.0 nM and 5.0 3.0 nM and 2.0 4.0 nM and 1.0 5.0 nM and 975 nM, 6.0 nM and 950 nM, 7.0 nM and 925 nM, 8.0 nM and 900 nM, 9.0 nM and 875 nM, 10.0 nM and 850 nM, 11.0 nM and 825 nM, 12.0 nM and 800 nM, 13.0 nM and 775 nM, 14.0 nm and 750 nM, 15.0 nM and 725 nM, 16.0 nM and 700 nM, 17.0 nM and 675 nM, 18.0 nM and 650 nM, 19.0 nM and 625 nM, 20.0 nM and 600 nM, 25 nM and 575 nM, 30 nM and 550 nM, 35 nM and 525 nM, 40 nM and 500 nM, 45 nM and 475 nM, 50 nM and 450 nM, 55 nM and 425 nM, 60 nM and 400 nM, 65 nM and 375 nM, 70 nM and 350 nM, 75 nM and 325 nM, 80 nM and 300 nM, 85 nM and 275 nM, 90 nM and 250 nM, 95 nM and 225 nM, 100 nM and 200 nM, 125 nM and 175 nM, 125 nM and 150 nM, and, 150 nM and 175 nM.

In some examples, the amount/concentration of LCO is effective to enhance the growth of the plant to which the composition is applied.

### Combinations and Uses

Disclosed are compositions and methods that use Bacillus amyloliquefaciens bacteria and LCOs. In one example, the Bacillus amyloliquefaciens may be strain SB 3281. Multiple Bacillus amyloliquefaciens strains may be combined. In one example, the LCOs may be Nod factors, Myc factors, or a combination thereof.

The Bacillus amyloliquefaciens and LCO components of the disclosed compositions may be formulated together (e.g., as a combination) and applied to plants at the same time, formulated separately and applied to plants at the same time (e.g., simultaneously), or formulated separately and applied to plants at different times (e.g., sequentially).

The compositions may be formulated for various agricultural applications (e.g., seed coating formulations, foliar applications, in-furrow applications, drench applications, etc.). The compositions described herein may be formulated with at least one additional agricultural excipient to achieve a particular purpose (e.g., to coat seeds, for foliar applications, for dilution, etc.). Non-limiting examples of agricultural excipients include carriers, polymers, wetting agents, surfactants, anti-freezing agents, and the like, and combinations thereof.

In some examples, the compositions further comprise at least one additional component selected from a fertilizer, plant hormone, antioxidant, plant growth-promoting bacterium (PGPB), pesticide, herbicide, insecticide, acaricide, gastropodicide, rodenticide, nematicide, fungicide, or virucides.

The compositions may be packaged into kits. In one example, a kit may contain a composition of Bacillus amyloliquefaciens, a composition of LCOs, and/or a combination of Bacillus amyloliquefaciens and LCOs. A kit may also contain instructions for using the enclosed compositions. Other embodiments of the kits may further comprise a means for applying the composition or compositions to a seed, or the site of germination, planting, or growth in soilless medium. In certain examples, the kits further comprise at least one fertiliser, plant hormone, antioxidant, plant growth-promoting bacteria (PGPB), pesticide, herbicide, insecticide, acaricide, gastropodicide, rodenticide, nematicide, fungicide, or virucide.

The compositions may be used in various methods. In one method, the compositions are used to improve plant growth. The composition may be applied to plants in a variety of ways. In one example, the compositions are applied to or exposed to a seed. There are various methods known for applying compositions to a seed, generally known as “coating” a seed. Generally, these methods involve contacting seeds with a liquid (but also can be dry; e.g., powder) formulation that contains the substances to be applied to the seeds. Over time, the liquid may dry on the surface of the seeds. The seeds then may be planted, for example, using soil compost, soilless medium, and the like. Compositions containing the Bacillus amyloliquefaciens and/or LCOs disclosed herein may also be exposed to a seed, for example, by planting seeds in a furrow and also delivering the bacteria and LCO compositions to the furrow (i.e., planting the seeds with the compositions). Delivering the compositions to a furrow may be facilitated using solid compositions of the bacteria and LCOs.

In some examples, the methods further comprise the step of planting the seed in soil, compost, or growing the seed in a soilless medium. In some examples, the methods further comprise the step of germinating the seed. In a further example, the seed is germinated prior to the step of planting or growing the seed in a soilless medium. In some examples, the seed or germinated seed is planted in a furrow. In other examples, the methods further comprise providing the seed or germinated seed with at least one of water, oxygen, or nutrients. In one example, the seed is germinated prior to the step of planting or growing the seed in a soilless medium. In some embodiments, the seed or germinated seed is planted in a furrow.

In one example, the seeds may be from a monocotyledonous or dicotyledonous plant species. In one example, the monocotyledonous plant seeds may be corn, rye, oat, millet, sugar cane, sorghum, wheat, rice, and the like. In one example, the dicotyledonous plant seeds may be tobacco, potato, tomato, bean, soybean, mustard, carrot, cassava, Arabidopsis, and the like.

### Effects of the Combinations

The effect of the applying the disclosed compositions to plants or using the disclosed methods, is generally to improve plant growth. Improvement of plant growth may be determined by comparing various parameters of plants to which the disclosed compositions have been applied to plants to which the disclosed compositions have not been applied. The comparing may occur at various times after the compositions have been applied, and after the plants have been allowed to grow.

A variety of different properties of plants may be measured or determined to determine whether the disclosed compositions or methods improve plant growth as compared to plants to which the compositions or methods have not been applied. In some examples the improved plant growth may be an increase in the dry weight of roots and/or shoots of a plant; and/or an increase in the dry weight of total plant biomass; and/or the average length of roots and/or shoots of a plant; and/or, the average number of roots and/or shoots produced by a plant, and/or an increase in the frequency of germination of a seed, and/or a decrease in the time taken for a seed to germinate, relative to a control plant or seed.

In some examples, the increases in the above-indicated parameters in plants that have been treated as disclosed herein, may be a 1% change as compared to plants that have not been treated. In some examples, the changes in the above-indicated parameters may be at least 3%, at least 5%, at least 7%, at least 9%, at least 11%, at least 13%, at least 15%, at least 17%, or at least 19%. In some examples, the changes in the above-indicated parameters may be between 1-20%, 2-19%, 3-18%, 4-17%, 5-16%, 6-15%, 7-14%, 8-13%, 9-12%, or 10-11%.

Not all of the above-indicated parameters may be affected by the disclosed compositions or methods. In various examples, 1, 2, 3, 4, 5, 6, 7, 8, 9, 10, or more parameters may be improved in the treated plants as compared to untreated plants. Generally, at least one of the parameters will be shown to be improved by the treatment, as compared to plants that have not been treated.

In one example, the effect of treating plants with the combination of Bacillus amyloliquefaciens and LCO produces a more than additive improvement in plant growth as compared to treating plants with the Bacillus amyloliquefaciens component alone or the LCO component alone. For example, for a given plant parameter, the sum of the effects on plant growth for plants treated with the Bacillus amyloliquefaciens component alone and the LCO component alone is less than the effect of the combination of Bacillus amyloliquefaciens and LCO (i.e., the disclosed combinations) on plant growth. In order to conclude that the disclosed combinations have a more than additive effect on plants, it is not required that all measured parameters show the more than additive effect. Generally, if at least one parameter shows the more than additive effect, one may conclude that the effect of the combination is more than additive. In one example, a parameter that shows this more than additive effect may be seedling dry weight or biomass, although any parameter may be used.

In one example, the effect of treating plants with the combination of Bacillus amyloliquefaciens and LCO produces an additive improvement in plant growth as compared to treating plants with the Bacillus amyloliquefaciens component alone or the LCO component alone. For example, for a given plant parameter, the sum of the effects on plant growth for plants treated with the Bacillus amyloliquefaciens component alone and the LCO component alone may generally be equal to the effect of the combination of Bacillus amyloliquefaciens and LCO (i.e., the disclosed combinations) on plant growth.

### EXAMPLE EMBODIMENTS OF THE INVENTION

1. A composition, comprising a Bacillus amyloliquefaciens and at least one lipochitooligosaccharide (LCO).
2. The composition of embodiment 1, where the Bacillus amyloliquefaciens includes an isolated and biologically pure culture.
3. The composition of embodiment 1, where the Bacillus amyloliquefaciens includes spores.
4. The composition of embodiment 1, where the Bacillus amyloliquefaciens includes a cell free supernatant of a medium in which the Bacillus amyloliquefaciens was cultured.
5. The composition of any of embodiments 1-4, where the Bacillus amyloliquefaciens includes strain SB3281 (ATCC # PTA-7542).
6. The composition any of embodiments 1-5, where the LCO is a Nod factor or a Myc factor.
7. The composition of embodiment 6, where the Nod factor includes LCO V (C18:1) having the structure as shown in FIG. 1A.
8. The composition of embodiment 6, where the Myc factor includes LCO IV (C16:0, S) having the structure as shown in FIG. 1B.
9. The composition of any of embodiments 1-8, where the composition further includes at least one additional component selected from a fertilizer, plant hormone, antioxidant, plant growth-promoting bacterium (PGPB), pesticide, herbicide, insecticide, acaricide, gastropodicide, rodenticide, nematicide, fungicide, or virucide.
10. A composition as defined in any of embodiments 1-10, for use in improving plant growth.
11. The composition for use according to embodiment 10, where the improved plant growth is an increase in the dry weight of roots and/or shoots of a plant; and/or, increase in seeding dry weight; and/or the average length of roots and/or shoots of a plant; and/or, the average number of roots and/or shoots produced by a plant, and/or an increase in the frequency of germination of a seed, and/or a decrease in the time taken for a seed to germinate, relative to a control plant or seed.
12. The composition for use according to embodiment 11, where the increase in the dry weight of roots and/or shoots is at least a 1% increase; and/or where the increase in seedling dry weight is at least a 1% increase; and/or where the increase in the average length of roots and/or shoots is at least a 1% increase; and/or where the increase in the average number of roots and/or shoots produced is at least a 1% increase, and/or where the increase in the frequency of germination of a seed is at least a 1% increase, and/or where the decrease in the time taken for a seed to germinate is at least a 1% decrease.
13. The composition for use according to embodiment 12, where the increase in the dry weight, average length of, or number of roots and/or shoots, the increase in seedling dry weight, the increase in the frequency of germination of a seed, or the decrease in the time taken for a seed to germinate, is between 1-20%, 2-19%, 3-18%, 4-17%, 5-16%, 6-15%, 7-14%, 8-13%, 9-12%, or 10-11%.
14. The composition for use according to any of embodiments 10-13, where the improvement in plant growth is a more than additive effect of the isolated and biologically pure culture of Bacillus amyloliquefaciens and the LCO.
15. The composition for use according to any of embodiments 10-13, where the improvement in plant growth is an additive effect of the isolated and biologically pure culture of Bacillus amyloliquefaciens and the at least one LCO.
16. The composition for use according to any of embodiments 10-15, where the plant includes corn.
17. A composition, comprising a Bacillus amyloliquefaciens, and a Nod factor or a Myc factor, the composition capable of more than additively improving corn plant growth when applied to a seed or exposed to a seed.
18. The composition according to embodiment 17, where more than additively improving corn plant growth includes a more than additive increase in corn seedling dry weight as compared to increases produced by Bacillus amyloliquefaciens alone, or Nod factor or Myc factor alone.
19. A method of improving plant growth, comprising applying to a seed, or exposing a seed to, a composition as defined in any of embodiments 1-9.
20. A method of improving plant growth, comprising:
   a) applying to a seed, or exposing a seed to, a composition including an isolated and biologically pure culture of Bacillus amyloliquefaciens, or spores from Bacillus amyloliquefaciens; and,
   b) applying to a seed, or exposing a seed to, a composition comprising at least one LCO.
21. The method according to embodiment 20, where the Bacillus amyloliquefaciens is strain 3281 (ATCC # PTA-7542).
22. The method according to embodiment 20 or embodiment 21, where the at least one LCO is a Nod factor or a Myc factor.
23. The method of embodiment 22, where the Nod factor includes LCO V (C18:1) having the structure as shown in FIG. 1A.
24. The method of embodiment 22, where the Myc factor includes LCO IV (C16:0, S) having the structure as shown in FIG. 1B.
25. The method according to any of embodiments 20-24, where steps a) and b) are carried out sequentially.
26. The method according to any of embodiments 20-24, where steps a) and b) are carried out simultaneously.
27. The method according to any of embodiments 20-26, where the method further includes the step of planting the seed in soil, compost, or growing the seed in a soilless medium.
28. The method of according to any of embodiments 20-27, where the method further includes the step of germinating the seed.
29. The method according to embodiment 28, where the seed is germinated prior to the step of planting or growing the seed in a soilless medium.
30. The method according to any of embodiments 27-29, where the seed or germinated seed is planted in a furrow.
31. The method according to any of embodiments 27-30, where the seed or germinated seed, is provided with at least one of water, oxygen, or nutrients.
32. The method according to any of embodiments 20-31, where the seed includes a corn seed.
33. The method according to any of embodiments 20-32, where the composition or compositions are applied to the seed.
34. The method according to embodiment 33, where the composition or compositions are applied by coating the seed.
35. The method according to any of embodiment 20-34, where the seed is exposed to the composition or compositions.
36. The method according to embodiment 35, where the seed is exposed at the site of germination, planting, or growth in a soilless medium.
37. The method according to any of embodiments 20-36, where the improved plant growth is an increase in the dry weight of roots and/or shoots of a plant; and/or the average length of roots and/or shoots of a plant; and/or the average number of roots and/or shoots produced by a plant, and/or an increase in the frequency of germination of a seed, and/or a decrease in the time taken for a seed to germinate; and/or an increase in the dry weight of total plant biomass, relative to a control plant or seed.
38. The method according to embodiment 37, where the increase in the dry weight of roots and/or shoots is at least a 1% increase; and/or where the increase in the average length of roots and/or shoots is at least a 1% increase; and/or, where the increase in the average number of roots and/or shoots produced is at least a 1% increase, and/or where the increase in the frequency of germination of a seed is at least a 1% increase, and/or where the decrease in the time taken for a seed to germinate is at least a 1% decrease, and/or where the increase in the dry weight of total plant biomass is at least a 1% increase.
39. The method according to embodiment 37, where the increase in the dry weight, average length of, or number of roots and/or shoots, the increase in the frequency of germination of a seed, the decrease in the time taken for a seed to germinate, or the increase in the dry weight of total plant biomass, is between 1-20%, 2-19%, 3-18%, 4-17%, 5-16%, 6-15%, 7-14%, 8-13%, 9-12%, or 10-11%.
40. The method according to any of embodiments 20-39, where the improvement in plant growth is a more than additive effect of the isolated and biologically pure culture of Bacillus amyloliquefaciens and the at least one LCO.
41. The method according to any of embodiments 20-39, where the improvement in plant growth is an additive effect of the isolated and biologically pure culture of Bacillus amyloliquefaciens and the at least one LCO.
42. A method of improving plant yield, comprising applying to a seed, or exposing a seed to, a composition as defined in any of embodiments 1-10.
43. A seed, where the seed has been exposed to, or has had applied to it, a composition comprising an isolated and biologically pure culture of Bacillus amyloliquefaciens strain 3281 (ATCC # PTA-7542).
44. A seed, wherein the seed has been exposed to, or has had applied to it, a composition according to any of embodiments 1-10.
45. The seed according to embodiment 43 or embodiment 44, where the seed is from a monocotyledonous or a dicotyledonous plant species.
46. The seed according to embodiment 45, where the monocotyledonous species is selected from the list consisting of corn, rye, oat, millet, sugar cane, sorghum, wheat and rice.
47. The seed according to embodiment 45, where the dicotyledonous species is selected from the list consisting of tobacco, potato, tomato, bean, soybean, mustard, carrot, cassava, and Arabidopsis.
48. The seed according to any of embodiments 43-47, where the seed is capable of producing a plant with improved growth.
49. A method of preparing a treated seed, comprising applying to a seed, or exposing a seed to, a composition as defined in any of embodiments 1-10.
50. A plant produced from a seed as defined in embodiments any of 43-48.
51. A kit for improving plant growth and/or improving plant yield, comprising:
   a) a composition as defined in any of embodiments 1-10; and,
   b) instructions for using the kit.
52. A kit for improving plant growth or improving plant yield, comprising:
   a) a composition comprising an isolated and biologically pure culture of Bacillus amyloliquefaciens strain SB3281 (ATCC # PTA-7542);
   b) a composition comprising at least one LCO; and,
   c) instructions for using the kit.
53. A kit according to embodiment 51 or embodiment 52, where the at least one LCO is a Nod factor or a Myc factor.
54. A kit according to embodiment 52, where the Nod factor includes LCO V (C18:1) having the structure as shown in FIG. 1A.
55. A kit according to embodiment 52, where the Myc factor includes LCO IV (C16:0, S) having the structure as shown in FIG. 1B.
56. A kit according to any of embodiments 55-61, where the kit further comprises a means for applying the compositions to a seed, or the site of germination, planting, or growth in a soilless medium.
57. A kit according to any of embodiments 51-56, where the kit further comprises at least one fertiliser, plant hormone, antioxidant, plant growth-promoting bacteria (PGPB), pesticide, herbicide, insecticide, acaricide, gastropodicide, rodenticide, nematicide, fungicide, or virucide.

### EXAMPLES

The following examples are for the purpose of illustrating various embodiments and are not to be construed as limitations.

### Example 1. Bacillus amyloliquefaciens Strain SB 3281 and Nod Factor LCO Effects on Corn

A study was performed to determine the effect of seed treatment of Bacillus amyloliquefaciens, a Nod factor, or a combination of Bacillus amyloliquefaciens and a Nod factor on corn growth. Corn seeds were treated with Bacillus amyloliquefaciens strain SB3281 bacteria alone, an LCO V (C18:1) Nod factor (FIG. 1A) alone, or a combination of the bacterial strain and Nod factor.

In some experiments, cell-free culture medium (i.e., called supernatant, obtained after centrifuging part of the culture and collecting the supernatant), in which Bacillus amyloliquefaciens had been cultured, was used instead of the bacteria. In some experiments, spores of Bacillus amyloliquefaciens were used instead of the bacteria.

Seeds treated as above were tested in a system designed to test various parameters of early seed germination. In this system, the seeds were placed on moist germination paper, in petri dishes, in a representative mock moist chamber experiment (for each seed treatment, 3 petri dishes, each with 5 seeds per dish, were used; 10 ml of deionized water was added to the germination paper per dish). The seeds were allowed to grow in the petri dishes at room temperature for approximately 2 weeks. After that time, various parameters of the seedlings were measured. The lengths of roots/shoots were measured in some experiments. In some experiments, the dry weights of the roots/shoots were determined after dissecting roots from shoots, drying the roots/shoots (placed in a coin envelope) in a 70° C. oven for 2 days, and weighing the roots/shoots. The individual biomass per seedling was determined from these measurements in order to find the average biomass per treatment.

To obtain the Bacillus amyloliquefaciens bacterium for the study, strain SB3281 (ATCC PTA-7542) was maintained on Standard Methods Agar (“SMA”; Smith River Biologicals; Ferrum, Va., U.S.). Single colonies were selected from SMA and used to inoculate 5 ml of LB medium. Twenty-four hour old LB cultures were used to inoculate a 250 ml flask containing 100 ml of Si medium (per liter contained 9.4 g of yeast extract, 9.4 g of corn syrup solids, 0.5 g of magnesium chloride hexahydrate, 0.2 g of manganese (II) chloride tetrahydrate, 0.2 g of calcium chloride dehydrate, and 0.27 g of sodium hydroxide, pH 7.0). The flask was incubated at 30° C. on a shaker at 165 rpm. Samples of the bacteria were taken from the culture at 7 days (T7). The number of bacteria in the cultures at the various time points was determined by plating serial dilutions from the cultures on SMA plates and the colonies that formed were counted and multiplied by the dilution factor. The number of spores in the cultures were determined by heating aliquots from the cultures at 80° C. in a water bath for 10 minutes to kill vegetative cells. Serial dilutions were plated as above and colony counts multiplied by the dilution factor. Generally, the cultures contained 10⁹ bacteria or spores per ml.

Corn seeds were treated by applying a total of 500 μl of treatment substances (e.g., bacteria, LCO, etc.) to 50 g of corn seeds (Monsanto, DKC 63-33). The seeds were allowed to dry at room temperature in bags for 3-4 hours before used in the moist chamber experiments. For treatment of seeds with a single substance (e.g., bacteria alone, LCO alone), 500 μl of the bacterial culture was used, or 500 μl of a LCO solution that contained 6 μl of a 10⁻⁸ M LCO stock solution in 100 ml of buffer was used. For treatment of seeds with two substances (e.g., bacteria and an LCO), 250 μl of the bacterial culture and 250 μl of the 6×10⁻¹³ M LCO stock solution was used. Negative control corn seeds were treated with 500 μl of water.

Data from example experiments of this type are shown below.

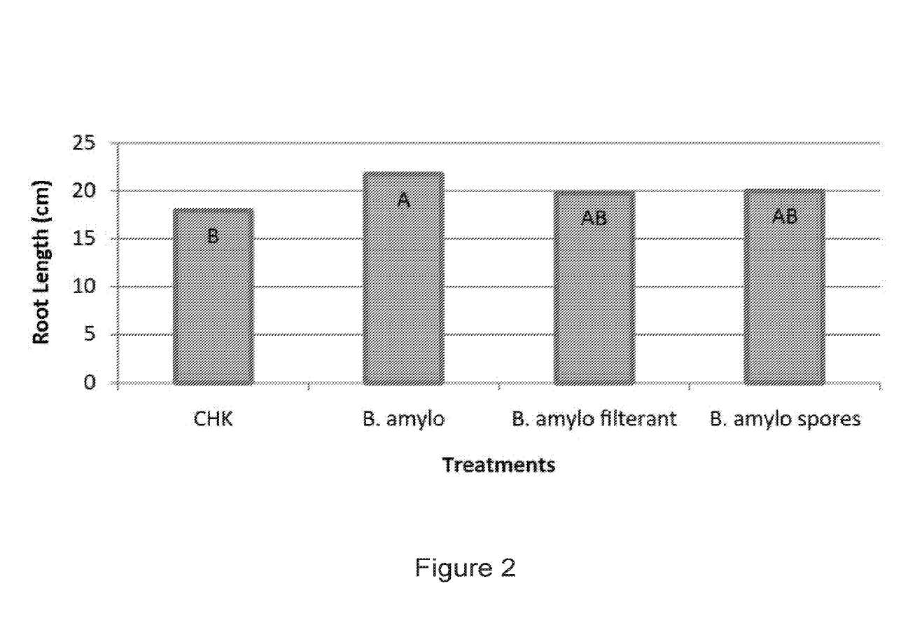

Table 1, below, and FIG. 2 show data, using Bacillus amyloliquefaciens grown for 7 days T7, in a moist chamber experiment that compared root length of germinated corn seeds treated with Bacillus amyloliquefaciens as compared to control cells that were treated with water.

TABLE 1
Effect of Bacillus amyloliquefaciens on corn root
length in moist chamber system

| | Root length | Standard | Percent increase |
|---|---|---|---|
| | (cm) and | error of | root length |
| Seed treatment | significance¹ | measurements | over control |
| Control | 17.894 | 1.076 | — |
| | B | | |
| Bacteria | 21.708 | 1.442 | 21.3 |
| | A | | |
| Bacterial | 19.767 | 1.507 | 10.5 |
| supernatant | AB | | |
| Bacterial spores | 19.936 | 1.211 | 11.4 |
| | AB | | |

¹Different connecting letters indicate a significant difference at p ≤ 0.05 (Student's t-test)

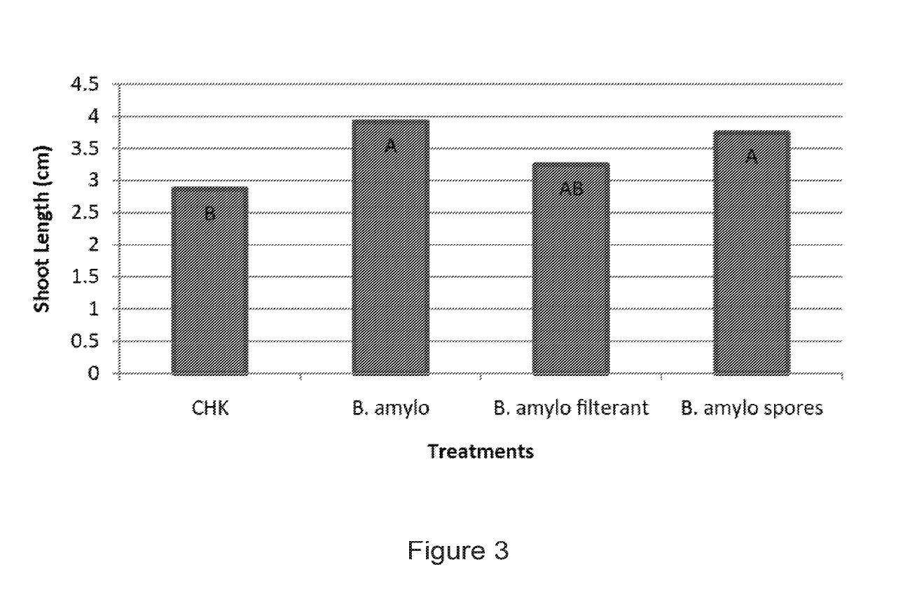

Table 2, below, and FIG. 3 show data, using Bacillus amyloliquefaciens grown for 7 days (T7), in a moist chamber experiment that compared shoot length of germinated corn seeds treated with Bacillus amyloliquefaciens as compared to control seeds that were treated with water.

TABLE 2
Effect of Bacillus amyloliquefaciens on corn shoot
length in moist chamber system

| | Shoot length | Standard | Percent increase |
|---|---|---|---|
| Seed | (cm) and | error of | shoot length |
| treatment | significance¹ | measurements | over control |
| Control | 2.867 B | 0.257 | — |
| Bacteria | 3.916 A | 0.378 | 36.6 |

¹Different connecting letters indicate a significant difference at p ≤ 0.05 (Student's t-test)

These data show that Bacillus amyloliquefaciens bacteria increased corn plant growth as measured by root length and shoot length in the assays. Bacterial supernatant from medium in which the cells were grown, and spores from the Bacillus amyloliquefaciens, also increased corn plant growth, although not as well as the bacteria themselves.

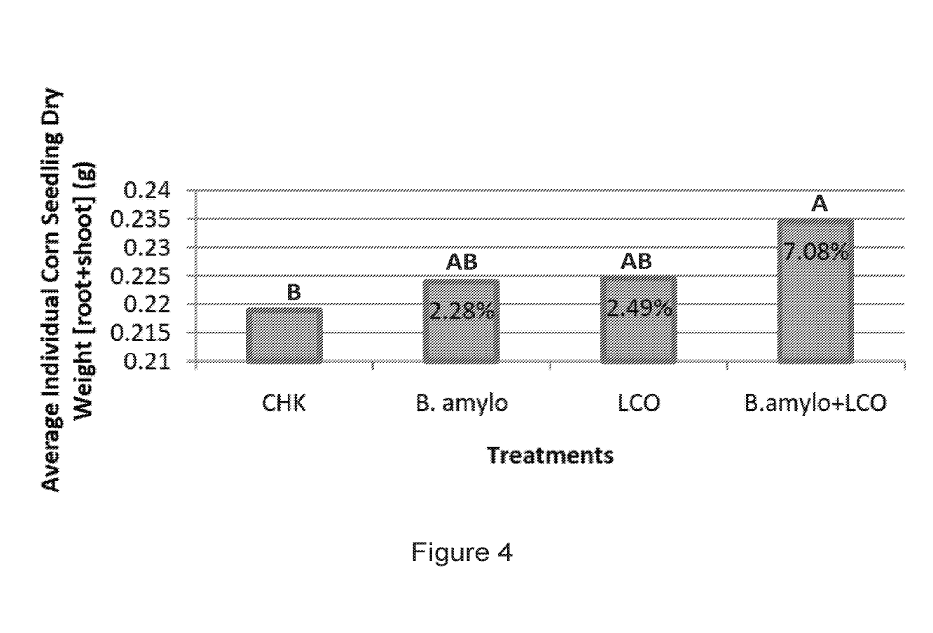

Table 3 and FIG. 4 show data, using Bacillus amyloliquefaciens grown for 7 days (T7), Nod factor, or Bacillus amyloliquefaciens and Nod factor, in a moist chamber experiment that compared corn seedling biomass for the different seed treatments.

TABLE 3
Effect of Bacillus amyloliquefaciens and Nod factor on
corn seedling dry weight in moist chamber system

| | Average | | |
|---|---|---|---|
| | individual | | Percent |
| | seedling | | increase |
| | dry weight | Standard | seedling |
| Seed | (g) and | error of | dry weight |
| treatment | significance¹ | measurements | over control |
| Control | 0.2190 | 0.0066 | — |
| | B | | |
| Bacteria | 0.2240 | 0.0087 | 2.28 |
| | AB | | |
| Nod factor | 0.2244 | 0.0044 | 2.47 |
| | AB | | |
| Bacteria + | 0.2345 | 0.0038 | 7.08 |
| Nod factor | A | | |

¹Different connecting letters indicate a significant difference at p ≤ 0.05 (Student's t-test)

These data show that Bacillus amyloliquefaciens alone and Nod factor alone, both increased corn plant growth as measured in this study. In this study, bacteria alone increased seedling dry weight 0.0050 g (0.2240-0.2190) or about 2.3%. Nod factor alone increased seedling dry weight 0.0054 g (0.2244-0.2190) or about 2.5%. Bacteria plus Nod factor increased seedling dry weight 0.0155 g (0.2345-0.2190) or about 7.1%. These data show that the combination of Bacillus amyloliquefaciens and Nod factor increased corn plant growth in a more than additive fashion, as compared to treatment of either Bacillus amyloliquefaciens alone or Nod factor alone.

### Example 2. Bacillus amyloliquefaciens Strain SB 3281 and Myc Factor LCO Effects on Corn

A study was performed to determine the effect of Bacillus amyloliquefaciens, LCOs, or combinations of Bacillus amyloliquefaciens and LCOs on corn growth. Corn seeds were treated with Bacillus amyloliquefaciens strain SB3281 bacteria alone, or in combination with an LCO IV (C16:0, S) Myc factor (FIG. 1B).

In this study, corn seeds treated with Bacillus amyloliquefaciens alone or Myc factor alone were compared to non-treated control corn seeds. In addition, corn seeds treated with Bacillus amyloliquefaciens and Myc factor were included in the study.

Seeds treated as above were tested in a system designed to test various parameters of early emergence of treated seeds. In this system, the treated seeds were planted in Metro-Mix® 830 (Sun Gro Horticulture; Agawan, Mass., U.S.) and grown in a growth chamber (25° C., 60% relative humidity, 18/6 hour light/dark cycle). Germination trays (12 rows of 6 wells) were filled with moist Metro-Mix® 830. Treated seeds were sown into the wells (30 wells per seed treatment) and grown in the growth chamber for 8 days.

The seedlings were harvested, and dry weights of roots/shoots from the seedlings were obtained. To measure the dry weight, the soil was removed from the roots, and dry weights of the roots/shoots and biomass per seedling were determined as described in Example 1.

To obtain the Bacillus amyloliquefaciens bacterium for the study, strain SB3281 (ATCC PTA-7542) was grown as described in Example 1. Samples of the bacteria were taken from the culture at 24 hrs (T1). The number of bacteria in the cultures at the various time points was determined by plating serial dilutions from the cultures on SMA plates and the colonies that formed were counted and multiplied by the dilution factor. The number of spores in the cultures were determined by heating aliquots from the cultures at 80° C. in a water bath for 10 minutes to kill vegetative cells. Serial dilutions were plated as above and colony counts multiplied by the dilution factor.

Corn seeds were treated by applying a total of 500 μl of treatment substances (e.g., bacteria, LCO, etc.) to 50 g of corn seeds (Monsanto, DKC 63-33). The seeds were allowed to dry at room temperature in bags for 3-4 hours before used in the Metro-Mix® experiments. Control seeds were treated with 500 μl of water. Another group of seeds was treated with 500 μl of a Bacillus amyloliquefaciens culture. A third group of seeds was treated with 500 μl of a 10⁻⁹ M solution of Myc factor. A fourth group of seeds was treated with 250 μl of the Bacillus amyloliquefaciens culture and 250 μl of a 10⁻⁹ M solution of Myc factor.

Data from example experiments of this type are shown below.

Table 4 shows data, using Bacillus amyloliquefaciens grown for 1 day (T1), in a Metro-Mix® 830 experiment that compared biomass of seedlings for the different seed treatments.

TABLE 4
Effect of Bacillus amyloliquefaciens and Myc factor on
corn seedling dry weight in Metro-Mix ® 830 system

| | Average | | |
|---|---|---|---|
| | individual | | Percent |
| | seedling | | increase |
| | dry weight | Standard | seedling |
| Seed | (g) and | error of | dry weight |
| treatment | significance¹ | measurements | over control |
| Control | 0.1985 | 0.0049 | — |
| | BC | | |
| Bacteria | 0.2072 | 0.0098 | 4.38 |
| | B | | |
| Myc factor | 0.1921 | 0.0028 | −3.22 |
| | C | | |
| Bacteria + | 0.2344 | 0.0188 | 18.09 |
| Myc factor | A | | |

¹Different connecting letters indicate a significant difference at p ≤ 0.05 (Student's t-test)

These data show that Bacillus amyloliquefaciens alone increased corn plant growth as measured in this study, while Myc factor alone appeared to have a negative effect on corn plant growth. In this study, bacteria alone increased seedling dry weight 0.0087 g (0.2072-0.1985) or about 4.4%. Myc factor alone decreased seedling dry weight 0.0064 g (0.1921-0.1985) or about −3.2%. Bacteria plus Myc factor increased seedling dry weight 0.0359 g (0.2344-0.1985) or about 18.1%. The data show that the combination of Bacillus amyloliquefaciens and Myc factor increased corn plant growth in a more than additive fashion, as compared to treatment of either Bacillus amyloliquefaciens alone or Myc factor alone.

While example compositions, methods, and so on have been illustrated by description, and while the descriptions are in considerable detail, it is not the intention to restrict or in any way limit the scope of the application. It is, of course, not possible to describe every conceivable combination of components or methodologies for purposes of describing the compositions, methods, and so on described herein. Additional advantages and modifications will readily appear to those skilled in the art. Therefore, the invention is not limited to the specific details and illustrative examples shown and described. Thus, this application is intended to embrace alterations, modifications, and variations that fall within the scope of the application. Furthermore, the preceding description is not meant to limit the scope of the invention.

To the extent that the term “includes” or “including” is employed in the detailed description or the claims, it is intended to be inclusive in a manner similar to the term “comprising” as that term is interpreted when employed as a transitional word in a claim. Furthermore, to the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use.

### Deposit of Biological Material

The following biological material has been deposited under the terms of the Budapest Treaty with the American Type Culture Collection, Manassas, Va. 20110-2209 USA on Apr. 20, 2006 or Aug. 18, 2006 by Novozymes Biologicals, Inc., and are identified as follows:

Bacillus amyloliquefaciens SB 3281—PTA-7542,

Bacillus amyloliquefaciens—PTA-7541,

Bacillus atrophaeus—PTA 7543,

Bacillus amyloliquefaciens—PTA 7544,

Bacillus amyloliquefaciens—PTA 7545,

Bacillus amyloliquefaciens—PTA 7546,

Bacillus subtilis subsp. Subtilis—PTA 7547,

Bacillus velezensis—PTA 7548,

Bacillus amyloliquefaciens—PTA 7549,

Bacillus simplex—PTA 7550,

Bacillus simplex—PTA 7789,

Bacillus amyloliquefaciens—PTA 7790,

Bacillus amyloliquefaciens—PTA 7791,

Bacillus atrophaeus—PTA 7792, and

Bacillus amyloliquefaciens—PTA 7793.

1. A composition, comprising a Bacillus amyloliquefaciens and at least one lipochitooligosaccharide (LCO).

2. The composition of claim 1, where the Bacillus amyloliquefaciens includes strain SB3281 (ATCC # PTA-7542).

3. The composition of claim 1, where the Bacillus amyloliquefaciens includes an isolated and biologically pure culture.

4. The composition of claim 1, where the Bacillus amyloliquefaciens includes spores.

5. The composition of claim 1, where the LCO is a Nod factor or a Myc factor.

6. The composition of claim 5, where the Nod factor includes LCO V (C18:1) having the structure as shown in FIG. 1A.

7. The composition of claim 5, where the Myc factor includes LCO IV (C16:0, S) having the structure as shown in FIG. 1B.

8. The composition of claim 1, where the composition is capable of more than additively improving corn plant growth when applied to a corn seed or exposed to a corn seed.

9. The composition of claim 8, where improving corn plant growth includes an increase in dry weight of roots or shoots of the corn plant; an increase in corn seeding dry weight; an increase in average length of roots or shoots of the corn plant; an increase in average number of roots or shoots produced by the corn plant; an increase in frequency of germination of the corn seed; or a decrease in time taken for the corn seed to germinate, relative to a control corn plant or corn seed.

10. The composition of claim 8, where more than additively improving corn plant growth includes a more than additive increase in corn seedling dry weight as compared to increases produced by Bacillus amyloliquefaciens alone, or Nod factor or Myc factor alone.

11. The composition of claim 1, where the composition further includes at least one additional component selected from a fertilizer, plant hormone, antioxidant, plant growth-promoting bacterium (PGPB), pesticide, herbicide, insecticide, acaricide, gastropodicide, rodenticide, nematicide, fungicide, or virucide.

12. A method of improving corn plant growth, comprising:
a) applying to a corn seed, or exposing a corn seed to, a composition including an isolated and biologically pure culture of Bacillus amyloliquefaciens, or spores from Bacillus amyloliquefaciens; and,
b) applying to the corn seed, or exposing the corn seed to, a composition comprising at least one LCO.

13. The method of claim 12, where the Bacillus amyloliquefaciens is strain 3281 (ATCC # PTA-7542).

14. The method of claim 12, where the at least one LCO is a Nod factor or a Myc factor.

15. The method of claim 14, where the Nod factor includes LCO V (C18:1) having the structure as shown in FIG. 1A.

16. The method of claim 14, where the Myc factor includes LCO IV (C16:0, S) having the structure as shown in FIG. 1B.

17. The method of claim 12, where the method further includes the step of planting the corn seed in soil, compost, or growing the corn seed in a soilless medium.

18. The method of claim 17, where the method further includes the step of germinating the corn seed.

19. The method of claim 12, where the improving corn plant growth is a more than additive effect of the isolated and biologically pure culture of Bacillus amyloliquefaciens and the at least one LCO.

20. The method of claim 19, where the improving corn plant growth includes an increase in dry weight of roots or shoots of the corn plant; an increase in corn seeding dry weight; an increase in average length of roots or shoots of the corn plant; an increase in average number of roots or shoots produced by the corn plant; an increase in frequency of germination of the corn seed; or a decrease in time taken for the corn seed to germinate, relative to a control corn plant or corn seed.
